(19) United States
(12) Patent Application Publication
(10) Pub. No.: US 2021/0163552 A1
(43) Pub. Date: Jun. 3, 2021

(54) PEPTIDE FOR INDUCING REGENERATION OF TISSUE, AND USE THEREOF

(71) Applicants: SHIONOGI & CO., LTD., Osaka-shi, Osaka (JP); StemRIM Inc., Ibaraki-shi, Osaka (JP)

(72) Inventors: Yoichiro NIHASHI, lbaraki-shi, Osaka (JP); Tomoyuki KAWACHI, Kobe-shi, Hyogo (JP); Yukiko YAMATSU, Hirakata-shi, Osaka (JP); Masataka FUMOTO, Suita-shi, Osaka (JP); Takehiko YAMAZAKI, Toyonaka-shi, Osaka (JP)

(73) Assignees: SHIONOGI & CO., LTD., Osaka-shi, Osaka (JP); StemRIM Inc., Ibaraki-shi, Osaka (JP)

(21) Appl. No.: 16/608,139

(22) PCT Filed: Apr. 24, 2018
(86) PCT No.: PCT/JP2018/016654

§ 371 Date: Oct. 24, 2019

(30) Foreign Application Priority Data
Apr. 25, 2017 (JP) 2017-085793

(51) Int. Cl. C07K 14/47; C12N 5/077

(52) U.S. Cl. CPC C07K 14/4702; A61K 38/00; C12N 5/0669

(57) ABSTRACT

A peptide which can be utilized as an active ingredient of a composition for use in stimulation of migration of PDGFRα-positive cells, a composition for use in recruitment of bone marrow mesenchymal stem cells from bone marrow to peripheral blood, a composition for use in regeneration of tissues, or a composition for use in stimulation of migration of bone marrow-derived stromal cells is provided.

### REFERENCE TO SEQUENCE LISTING SUBMITTED VIA EFS-WEB

This application includes an electronically submitted sequence listing in .txt format. The .txt file contains a sequence listing entitled “1422-0851PUS1 ST25.txt” created on Feb. 10, 2021 and is 195,846 bytes in size. The sequence listing contained in this .txt file is part of the specification and is hereby incorporated by reference herein in its entirety.

### TECHNICAL FIELD

The present invention relates to a peptide for inducing regeneration of tissues, and uses thereof.

### BACKGROUND ART

It has become even clearer that each of organs and tissues in the living bodies has tissue stem cells maintaining its structural and functional homeostasis. For example, the myocardial stem cells exist in the heart, the neural stem cells exist in the brain, and the epidermal stem cells and follicular stem cells exist in the skin, which supply myocardial cells, neural cells, epidermal cells, and follicular epithelial cells over the lifetime to maintain the structures and functions of the heart, the brain, and the skin. Meanwhile, the hematopoietic stem cells that differentiate into blood cells such as red blood cells, white blood cells, and platelets exist in the bone marrow.

These blood cells derived from the hematopoietic stem cells circulate all sorts of organs and tissues in the bodies through blood streams, and play some indispensable functions for life maintenance such as oxygen supply, immune responses, and hematostatis or recovery of damaged tissues. In other words, it is fair to say that the bone marrow hematopoietic stem cells contribute to the maintenance of homeostasis of all the tissues in the live bodies via peripheral circulation, rather than the maintenance of homeostasis of the bone marrow or the bone tissues in which the bone marrow hematopoietic stem cells are localized.

In the recent years, it has been clarified that in addition to the hematopoietic stem cells, the mesenchymal stem cells that are differentiable into not only mesodermal tissues of bones, cartilages, adipose or the like, but also ectodermal tissues such as nerves and epidermides exist in the bone marrow; however, the existing significance in the live bodies has not yet been clarified in many ways. However, if the hematopoietic stem cells that maintain homeostasis of all the tissues and organs by supplying blood cells via the peripheral circulation exist in the bone marrow, there is similarly an expected possibility that the mesenchymal stem cells that exist in the bone marrow would also contribute to the maintenance of homeostasis of the tissues in the live bodies by providing cells that can differentiate into bones, cartilages, adipose, nerves, epithelia, or the like via the peripheral circulation to the tissues or organs in the live bodies in need thereof.

Currently, regenerative medicine has been actively developed, in which bone marrow mesenchymal stem cells are prepared by collection of the bone marrow blood, expanded by cell culture, and grafted to a damaged site of the intractable tissues or into the peripheral circulation blood to induce the regeneration of damaged tissues. The clinical applications of transplantation of the bone marrow mesenchymal stem cells have been already been underway in the regenerative medicine of cerebral infarction, myocardial infarction, intractable skin ulcer or the like. Further, it has been clarified that the transplanted bone marrow mesenchymal stem cells exhibit an action of suppressing inflammations and immune responses and an action of suppressing fibrous scar formation locally in the live bodies, so that the clinical tests of the bone marrow mesenchymal stem cells transplantation therapy have been started as a new therapeutic method for graft versus host disease (GVHD), which is a serious side reaction after the bone marrow transplantation or transfusion, or for scleroderma, which is an autoimmune disease. However, the bone marrow blood containing the bone marrow mesenchymal stem cells is only collected by an invasive method in which thick needles are repeatedly pierced to the iliac bone. In addition, the bone marrow mesenchymal stem cells would gradually lose proliferation ability and pluripotency if the subculture is continued ex vivo. Further, the culture of the bone marrow mesenchymal stem cells based on the high-quality management that ensures safety of the in vivo transplantation would necessitate a specialized culturing equipment such as CPC (cell processing center), so that the cell culture is enabled at very limited universities or business enterprises at present. In other words, in order to make the regenerative medicine using the bone marrow mesenchymal stem cells available to many patients in the world suffering from intractable tissue damages, it is an urgent task to develop techniques for the regenerative medicine with the mesenchymal stem cells to be enabled in all sorts of medical facilities.

HMGB1 (High mobility group box 1: high mobility group box 1 protein) was identified about 30 years ago as a non-histone chromatin protein that controls the gene expression or repair of DNA by controlling an intranuclear chromatin structure. The structure of the HMGB1 protein is constituted mainly by two DNA binding domains, in which a DNA binding domain at an N-terminal side is called an A-box, and a DNA binding domain at a C-terminal side is called a B-box. According to the past studies, it has been clarified that a domain that binds to TLR in the HMGB1 molecule and evokes an inflammatory reaction exists within the B-box.

It has been reported that the PDGFRα-positive cells are recruited in the blood from the bone marrow by administering mice generated with skin ulcer or cerebral infarction with a recombinant HMGB1 protein from tail vein, and further the cells are accumulated in the skin ulcer portion or the cerebral infarction portion, thereby strongly inducing the regeneration of skin ulcer or cerebral infarction. It has been already reported that the PDGFRα-positive cells in the bone marrow are mesenchymal stem cells that are differentiable into bones, cartilages, adipose, and further to nerves or epithelia. In other words, the PDGFRα-positive mesenchymal stem cells in the bone marrow are recruited to peripheral circulation by the HMGB1 administration, thereby making it possible to accumulate many of the mesenchymal stem cells in the damaged tissues in the live bodies without extracorporeally taking the cells, and subjecting them to a specialized culturing.

The regeneration-inducing medicine can be enabled with the bone marrow mesenchymal stem cells in all sorts of medical facilities by developing HMGB1 as a medicament for inducing regeneration of damaged tissues by the recruitment in blood of the bone marrow mesenchymal stem cells in the live bodies. As a result, many of the disadvantages faced in the present regenerative medicine using the bone marrow mesenchymal stem cells would be overcome.

As mentioned above, the HMGB1 medicament is an epoch-making therapeutic agent which promotes the recruitment of the bone marrow mesenchymal stem cells in blood or the accumulation to damaged tissues, thereby inducing regeneration of tissues in the body. Regarding HMGB1, it has been already reported that not only a full length of HMGB1 protein but also fragments of the HMGB1 protein has a similar action.

### PRIOR ART REFERENCES

### Patent Publications

Patent Publication 1: WO 2008/053892

Patent Publication 2: WO 2007/015546

Patent Publication 3: WO 2009/133939

Patent Publication 4: WO 2009/133943

Patent Publication 5: WO 2009/133940

Patent Publication 6: Japanese Unexamined Patent Publication No. 2005-537253 Patent Publication 7: WO 2012/147470

### Non-Patent Publications

Non-Patent Publication 1: Bustin et al., Mol Cell Biol, 19:5237-5246, 1999
Non-Patent Publication 2: Hori et al., J. Biol. Chem., 270, 25752-25761, 1995
Non-Patent Publication 3: Wang et al., Science, 285:248-251, 1999
Non-Patent Publication 4: Muller et al., EMBO J, 20:4337-4340, 2001
Non-Patent Publication 5: Wang et al., Science, 285:248-251, 1999
Non-Patent Publication 6: Germani et al., J Leukoc Biol. Jan; 81(1):41-5, 2007
Non-Patent Publication 7: Palumbo et al., J. Cell Biol., 164:441-449, 2004
Non-Patent Publication 8: Merenmies et al., J. Biol. Chem., 266:16722-16729, 1991
Non-Patent Publication 9: Wu Y et al., Stem cells, 25:2648-2659, 2007
Non-Patent Publication 10: Tamai et al., Proc Natl Acad Sci USA. 2011 Apr. 4. [Epub ahead of print], 108:6609-6614, 2011
Non-Patent Publication 11: Yang et al., J Leukoc Biol. January; 81(1):59-66, 2007

### SUMMARY OF THE INVENTION

### Problems to be Solved by the Invention

An object to be solved by the present invention is to provide a peptide having activity of migrating bone marrow-derived stromal cells, in other words, a peptide for inducing regeneration of tissues.

### Means to Solve the Problems

As a result of intensive studies, the present inventors have found out that a peptide having substitution of an amino acid of position 43 and/or position 44 in the peptide consisting of amino acids of position 1 to position 44 of the HMGB1 protein, and a peptide having substitution of an amino acid of position 43 and/or position 44 in the peptide consisting of the amino acids of position 2 to position 44 of the HMGB1 protein have the activity of migrating stromal cells derived from murine bone marrow, and the present invention has been perfected thereby.

Concretely, the present invention relates to:

(1) a peptide selected from the group consisting of:

a peptide having the amino acid sequence shown in any one of SEQ ID NOs: 1 to 399 and 403,

a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 399 and 403, and having an activity of stimulating migration of cells,

a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 399 or an amino acid of position 1 to position 41 of the peptide having the amino acid sequence shown in SEQ ID NO: 403, and having an activity of stimulating migration of cells, a derivative thereof, a pharmaceutically acceptable salt thereof, or a solvate thereof;

(2) a peptide selected from the group consisting of:

a peptide having the amino acid sequence shown in any one of SEQ ID NOs: 1 to 5, 126, 128, 129, 138, 167, 248, 254, 280, 282, 283, 292 and 403,

a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5, 126, 128, 129, 138, 167, 248, 254, 280, 282, 283, 292 and 403, and having an activity of stimulating migration of cells, and

a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5, 126, 128, 129, 138, 167, 248, 254, 280, 282, 283 and 292, or an amino acid of position 1 to position 41 of the peptide having the amino acid sequence shown in SEQ ID NO: 403, and having an activity of stimulating migration of cells, a derivative thereof, a pharmaceutically acceptable salt thereof, or a solvate thereof;

(3) a peptide selected from the group consisting of:

a peptide having the amino acid sequence shown in any one of SEQ ID NOs: 1 to 5, 167 and 403,

a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5, 167 and 403, and having an activity of stimulating migration of cells, and

a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5 and 167 or at an amino acid of position 1 to position 41 of the peptide having the amino acid sequence shown in SEQ ID NO: 403, and having an activity of stimulating migration of cells,

a derivative thereof, a pharmaceutically acceptable salt thereof, or a solvate thereof;
(4) a peptide selected from the group consisting of:

a peptide having the amino acid sequence shown in any one of SEQ ID NOs: 1 to 5 and 403,

a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5 and 403, and having an activity of stimulating migration of cells, and

a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5 or at an amino acid of position 1 to position 41 of the peptide having the amino acid sequence shown in SEQ ID NO: 403, and having an activity of stimulating migration of cells, a derivative thereof, a pharmaceutically acceptable salt thereof, or a solvate thereof;

(5) a composition for use in stimulation of migration of PDGFRα-positive cells, containing a peptide, a derivative thereof, a pharmaceutically acceptable salt thereof, or a solvate thereof as defined in any one of the above (1) to (4);
(6) a composition for use in recruitment of bone marrow mesenchymal stem cells from bone marrow to peripheral blood, containing a peptide, a derivative thereof, a pharmaceutically acceptable salt thereof, or a solvate thereof as defined in any one of the above (1) to (4);
(7) a composition for use in regeneration of tissues, containing a peptide, a derivative thereof, a pharmaceutically acceptable salt thereof, or a solvate thereof as defined in any one of the above (1) to (4); and
(8) a composition for use in stimulation of migration of bone marrow-derived stromal cells, containing a peptide, a derivative thereof, a pharmaceutically acceptable salt thereof, or a solvate thereof as defined in any one of the above (1) to (4).

### Advantageous Effects of the Invention

The peptide of the present invention can be utilized as an active ingredient of, for example, a composition for use in stimulation of migration of PDGFRα-positive cells, a composition for use in recruitment of bone marrow mesenchymal stem cells from bone marrow to peripheral blood, a composition for use in regeneration of tissues, or a composition for use in stimulation of migration of bone marrow-derived stromal cells.

### BRIEF DESCRIPTION OF THE DRAWINGS

FIG. 1 is a graph showing the results of the stability test of the peptides in plasma for six kinds of peptides (a peptide of position 2 to position 44 of human HMGB1 (2-44), and five kinds of peptides in which amino acids at position 43 or/and position 44 are modified (Peptide-1 to -5)). P1 to P5 mean the peptides of Peptide-1 to -5. The axis of ordinates is the remaining rate when the peptide immediately after the addition of the plasma was calculated as 100, and the axis of the abscissas is the time after the addition of the plasma. Here, the sample of 15 minutes or later after the addition of the plasma of the peptide of 2-44 was lower than the quantifiable lower limit (0.5 μg/mL), so that the remaining rate could not be plotted in FIG. 1.

FIG. 2 is a graph of the results of the measurements of the cell-migration activity of two kinds of peptides (a peptide of position 2 to position 44 of human HMGB1 (2-44), and a peptide in which the amino acids at position 43 and position 44 are modified (Peptide-6)). NC is a negative control.

FIG. 3 is a graph showing the results of the stability test in plasma of three kinds of peptides (a peptide of position 2 to position 44 of human HMGB1 (2-44), and two kinds of peptides in which amino acids at position 43 and position 44 are modified (Peptide-3 and -6)). The axis of ordinates is the remaining rate when the peptide immediately after the addition of the plasma was calculated as 100, and the axis of the abscissas is the time after the addition of the plasma.

### MODES FOR CARRYING OUT THE INVENTION

The present invention provides a peptide as defined in any one of (a) to (c) given below:

(a) a peptide having the amino acid sequence shown in any one of SEQ ID NOs: 1 to 399 and 403;

(b) a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 399 and 403, and having an activity of stimulating migration of cells; and

(c) a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 399 or an amino acid of position 1 to position 41 of the peptide having the amino acid sequence shown in SEQ ID NO: 403, and having an activity of stimulating migration of cells.

The present invention provides a composition for use in stimulation of migration of PDGFRα-positive cells or bone marrow-derived stromal cells, containing a peptide as defined in (a) to (c) given below:

(a) a peptide having the amino acid sequence shown in any one of SEQ ID NOs: 1 to 399 and 403;

(b) a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 399 and 403, and having an activity of stimulating migration of cells; and

(c) a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 399 or an amino acid of position 1 to position 41 of the peptide having the amino acid sequence shown in SEQ ID NO: 403, and having an activity of stimulating migration of cells.

The composition for use in stimulation of migration of PDGFRα-positive cells or bone marrow-derived stromal cells of the present invention includes a reagent composition and a pharmaceutical composition. The reagent composition as used herein is also expressed as a reagent, and the pharmaceutical composition is also expressed as a medicament, a pharmaceutical agent, or a pharmaceutical composition.

The reagent composition for use in stimulation of migration of PDGFRα-positive cells or bone marrow-derived stromal cells of the present invention can be used as, for example, reagents that are necessary for basic researches and clinical researches in the developments of regenerative medicine and regeneration-inducing medicine. For example, cells are recruited in tissues in the live bodies in experimental animals by the use of the reagent composition, thereby making it possible to study the levels of tissue repair and tissue function reconstruction. Also, for example, the researches on inducing regeneration of tissues can be carried out by the recruitment of the cells in vitro by the use of the reagent composition.

In addition, the pharmaceutical composition for use in stimulation of migration of PDGFRα-positive cells or bone marrow-derived stromal cells of the present invention can be used as, for example, a medicament in the regenerative medicine and the regeneration-inducing medicine. For example, the tissues can be regenerated by the use of the pharmaceutical composition. Also, for example, the pharmaceutical composition can prevent the lowering of the functions of tissues and organs by the decrease in the tissue stem cells as a so-called prophylactic medicament, or can delay the progress of aging changes as an anti-aging medicament.

Here, the composition for use in stimulation of migration of PDGFRα-positive cells or bone marrow-derived stromal cells as used herein is also expressed as an agent for use in stimulation of migration of PDGFRα-positive cells or bone marrow-derived stromal cells, a stimulation agent for cell migration, a composition for use in induction of cell migration, an agent for use in induction of cell migration, an inducing agent for cell migration, or an inducing agent of cells.

In the present invention the cell migration-stimulating activity means an activity of stimulating the cell migration. The cell migration-stimulating activity as used herein is also expressed as cell migration-inducing activity or cell-inducing activity.

The present invention provides a composition for use in recruitment of bone marrow mesenchymal stem cells from bone marrow to peripheral blood, containing a substance as defined in any one of the following (a) to (c):

(a) a peptide having the amino acid sequence shown in any one of SEQ ID NOs: 1 to 399 and 403;

(b) a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 399 and 403, and having an activity of stimulating migration of cells; and

(c) a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 399 or an amino acid of position 1 to position 41 of the peptide having the amino acid sequence shown in SEQ ID NO: 403, and having an activity of stimulating migration of cells.

The composition for use in recruitment of the bone marrow mesenchymal stem cells from bone marrow to peripheral blood of the present invention includes a reagent composition and a pharmaceutical composition.

The reagent composition for use in regeneration of tissues of the present invention can be used as, for example, reagents that are necessary for basic researches and clinical researches in the developments of regenerative medicine and regeneration-inducing medicine. In addition, the pharmaceutical composition for use in regeneration of tissues of the present invention can be used as, for example, a medicament in the regenerative medicine and the regeneration-inducing medicine. For example, the bone marrow cells are recruited to the peripheral circulation by the use of the pharmaceutical composition, so that the tissues can be regenerated. Also, for example, it is possible that the cells which are recruited to the peripheral blood by utilizing the pharmaceutical composition are collected extracorporeally, concentrated, and administered to tissues to carry out the treatment. In the conventional methods, since the cells are collected from the bone marrow at the deep part in the body, the live bodies are subjected to invasiveness. However, when the pharmaceutical composition of the present invention is used, the bone marrow cells are collected from the peripheral blood at low invasion, which can be utilized in bone marrow cell transplantation or the like. Here, the composition for use in recruitment of the bone marrow mesenchymal stem cells from bone marrow to peripheral blood is also expressed as a composition for use in induction of the bone marrow mesenchymal stem cells from bone marrow to peripheral blood.

The present invention provides a composition for use in regeneration of tissues, containing a substance as defined in any one of the following (a) to (c):

(a) a peptide having the amino acid sequence shown in any one of SEQ ID NOs: 1 to 399 and 403;

(b) a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 399 and 403, and having an activity of stimulating migration of cells; and

(c) a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 399 or an amino acid of position 1 to position 41 of the peptide having the amino acid sequence shown in SEQ ID NO: 403, and having an activity of stimulating migration of cells.

The composition for use in regeneration of tissues of the present invention includes a reagent composition and a pharmaceutical composition.

The reagent composition for use in regeneration of tissues of the present invention can be used as, for example, reagents that are necessary for basic researches and clinical researches in the developments of regenerative medicine and regeneration-inducing medicine. In addition, the pharmaceutical composition for use in regeneration of tissues of the present invention can be used as, for example, a medicament in the regenerative medicine and the regeneration-inducing medicine.

Here, the composition for use in regeneration of tissues as used herein is also expressed as a composition for use in induction or promotion of regeneration of tissues, an agent for use in induction or promotion of regeneration of tissues, a tissue regeneration-inducing agent or a tissue regeneration-promoting agent. In addition, the regeneration of tissues includes tissue repair.

The composition for use in regeneration of tissues of the present invention can be administered or added to any sites. In other words, the composition can exhibit its effects even when administered to any of tissues such as tissues in need of regeneration, tissues different from the tissues in need of regeneration, in blood, or the like. For example, when the composition is administered or added, the cells are recruited to a site to be administered or to be added or a tissue in a vicinity thereof, thereby inducing or promoting the regeneration of tissues. Also, for example, when the composition is administered or added to a damaged tissue site or a vicinity thereof, the cells are recruited in the damaged tissues, thereby inducing or promoting the regeneration of tissues. In addition, for example, when the composition is administered or added to a tissue different from the tissues in need of regeneration, the bone marrow cells are recruited from the bone marrow to the tissues in need of regeneration via the peripheral circulation, thereby inducing or promoting the regeneration of tissues. Here, the “peripheral circulation” is also called “blood circulation” or “peripheral circulation bloodstream.”

The tissues in need of regeneration include damaged tissues, necrotic tissues, tissues after surgery, tissues with reduced function, fibrotic tissues, aged tissues, diseased tissues, and the like. Examples include live body skin tissues and tissues from internal biopsy (surgery) (brain, lungs, heart, liver, stomach, small intestine, large intestine, pancreas, kidneys, urinary bladders, spleens, uterus, testis, blood, etc.).

The administration to the tissues different from the tissues in need of regeneration means administration to a site other than a site in need of regeneration (site different from the site in need of regeneration). Therefore, the “tissues different from the tissues in need of regeneration” can be expressed as a site different from the tissues in need of regeneration, a site different from a site in need of regeneration, a site away from the tissues in need of regeneration, a site away from a site in need of regeneration, a site distal from a site in need of regeneration, a site distal from the tissues in need of regeneration, a distal site, or a distal tissue. In other words, the composition of the present invention is effectively used in regeneration of tissues (brain, heart, etc.) that are difficult to be directly administered with a pharmaceutical agent from outside of the body.

The cells recruited to the tissues in need of regeneration differentiate into various cells to contribute to functional regeneration of the tissues in need of regeneration and to maintenance of functions or enhancement of functions of the tissues. In the present invention, the tissues in need of regeneration include tissues damaged by various pathologies caused by ischemic, hypoperfusive or hypoxic conditions, trauma, burns, inflammation, autoimmunity, genetic abnormalities, and the like, without being limited to these causes.

The tissues in the present invention are not particularly limited so long as they are tissues into which the bone marrow-derived cells can differentiate, which include, for example, all the tissues in the live bodies, such as skin tissues, bone tissues, cartilage tissues, muscle tissues, adipose tissues, cardiomuscular tissues, nervous system tissues, pulmonary tissues, digestive tract tissues, hepatic, biliary, and pancreatic tissues, and urogenital organs. In addition, it is made possible to carry out the treatment of inducing functional tissue regeneration in not only a cutaneous disease such as intractable skin ulcers, skin wounds, hydroa/bullous disease, or alopecia, but also in tissues in need of regeneration of cerebral infarction, myocardial infarction, bone fracture, pulmonary infarction, gastric ulcer, enteritis, or the like by the use of the above composition. The animal species to be administered with the above composition is not particularly limited, and includes mammals, birds, fish, and the like. The mammals include human or non-human animals, including, for example, human, mice, rats, monkeys, pigs, dogs, rabbits, hamsters, guinea pigs, horses, sheep, whales, and the like, but not particularly limited thereto.

In addition, the tissues different from the tissues in need of regeneration include blood tissues, muscular tissues, subcutaneous tissues, intradermal tissues, abdominal cavity, and the like.

The neural tissues include central nervous tissues, but not limited thereto. Also, the composition for use in regeneration of neural tissues can be used, for example, in the treatment of cerebral infarction, cerebral hemorrhage, brain contusion or the like, but not limited thereto. In addition, the composition for use in regeneration of bone tissues can be used, for example, in the treatment of bone fracture, but not limited thereto. Also, the composition for use in regeneration of skin tissues can be used, for example, in the treatment of skin ulcers, insufficient suture closure of surgical wounds, burns, cuts, bruises, skin erosion, abrasions, or the like, but not limited thereto.

In addition, in the present invention, the cells that are stimulated to migrate or the cells that are recruited from bone marrow to peripheral blood include undifferentiated cells and cells in different differentiation stages, but not limited thereto. Also, in the present invention, the cells that are stimulated to migrate or the cells that are recruited from bone marrow to peripheral blood include stem cells, non-stem cells, and the like, but not limited thereto. The stem cells include circulatory stem cells or non-circulatory stem cells. The non-circulatory stem cells include tissue stem cells that reside in the tissues. The circulatory stem cells include circulatory stem cells in blood.

Also, the cells that are stimulated to migrate or the cells that are recruited from bone marrow to peripheral blood include bone marrow-derived cells or hematopoietic stem cells, but not limited thereto. The “hematopoietic stem cells” as used herein are stem cells that can differentiate into blood cells such as red blood cells, platelets, mast cells, and dendritic cells, as well as white blood cells such as neutrophils, eosinophils, basophils, lymphocytes, monocytes, and macrophages. The markers thereof have been known to be CD34-positive, CD133-positive in human, CD34-negative, c-Kit-positive, Sca-1-positive, lineage marker-negative in mice. In addition, the hematopoietic stem cells have the feature that when cultured in a culture dish, it is difficult to culture alone, so that it is necessary to be co-cultured with stromal cells.

The term “bone marrow cells” as used herein means cells that are present in the bone marrow, while the term “bone marrow-derived cells” means “bone marrow cells” recruited or taken out from bone marrow to outside the bone marrow. The term “bone marrow cells” includes cells containing tissue progenitor cell population that are present in the bone marrow. In addition, the term “bone marrow-derived cells” may be cells containing mesoangioblasts, or may be cells excluding the mesoangioblasts.

The tissue progenitor cells are defined as undifferentiated cells having a unidirectional differentiability into specified tissue cells other than the blood system, and include undifferentiated cells having differentiability into mesenchymal tissues, epithelial tissues, neural tissues, parenchymatous organs, and vascular endothelia mentioned above.

The terms “bone marrow cells” and “bone marrow-derived cells” are hematopoietic stem cells and differentiated cells derived therefrom such as white blood cells, red blood cells, platelets, osteoblasts, and fibrocytes, or are stem cells which are represented by cells which have been hitherto called bone marrow mesenchymal stem cells, bone marrow mesenchymal stromal cells, bone marrow pluripotent stromal cells, or bone marrow pluripotent stem cells. The term “bone marrow stem cells” as used herein means stem cells existing in the bone marrow, and on the other hand, the term “bone marrow-derived stem cells” means “bone marrow stem cells” that are recruited or taken out from the bone marrow to outside of the bone marrow. In the present invention, the cells that are stimulated to migrate or the cells that are recruited from bone marrow to peripheral blood include “bone marrow-derived stem cells,” but not limited thereto. The “bone marrow cells” and the “bone marrow-derived cells” can be isolated by collection from bone marrow (collection of bone marrow cells), or peripheral blood collection. The hematopoietic stem cells are non-adherent cells, and some of the “bone marrow cells” and the “bone marrow-derived cells” are obtained as adherent cells by means of a cell culture of a monocyte fraction in blood obtained by collection from bone marrow (collection of bone marrow cells), or peripheral blood collection.

In addition, the “bone marrow cells” and the “bone marrow-derived cells” include mesenchymal stem cells, and preferably have a potential to differentiate into osteoblasts (which can be specified by observing calcium deposition when inducing differentiation), chondrocytes (which can be specified by Alcian blue staining-positive, safranin-O staining positive, etc.), adipocytes (which can be specified by Sudan III staining positive), and further mesenchymal cells such as fibroblasts, smooth muscle cells, stromal cells, tendon cells, further neural cells, epithelial cells (e.g., epidermal keratinocytes and intestinal epithelial cells expressing cytokeratin family), and vascular endothelial cells. The differentiated cells are not limited to the above cells, and also include the potential to differentiate into cells of parenchymatous organs such as liver, kidneys, and pancreas.

The term “bone marrow stromal cells” as used herein refer to cells other than the hematopoietic stem cells and the blood cells derived therefrom among the cells that exist in the bone marrow, which are collected from the bone marrow and capable of being cultured and proliferated as adherent cells on a culture dish. The bone marrow stromal cells or the bone marrow-derived stromal cells that are taken outside of the bone marrow include cells having pluripotency (bone marrow pluripotent stromal cells or bone marrow-derived pluripotent stromal cells). In addition, the bone marrow-derived stromal cells may be established as a cell line. Examples of the bone marrow-derived stromal cells in cell line include ST2 cells (Ogawa et al., EMBO J 1988; 7:1337-43), and the like. The ST2 cells are bone marrow-derived pluripotent stromal cell lines having a differentiating ability into osteoblasts and adipocytes.

The term “bone marrow mesenchymal stem cells” as used herein is used interchangeably with the terms “bone marrow mesenchymal stromal cells,” “bone marrow pluripotent stromal cells,” and “bone marrow pluripotent stem cells.” The “bone marrow mesenchymal stem cells” as used herein refer to cells that exist in the bone marrow, which are collected directly from the bone marrow, or indirectly from other tissues (blood, skin, adipose, and other tissues), and can be cultured and proliferated as adherent cells on a culture dish (made of plastic or glass). The cells have the characteristics of having a potential to differentiate into mesenchymal tissues such as bones, cartilages, and adipose (mesenchymal stem cells), or into skeletal muscles, heart muscles, and further neural tissues, and epithelial tissues (pluripotent stem cells), and can be obtained by collection of bone marrow cells.

On the other hand, the “bone marrow-derived mesenchymal stem cells,” the “bone marrow-derived mesenchymal stromal cells,” the “bone marrow-derived pluripotent stromal cells,” or the “bone marrow-derived pluripotent stem cells” that are recruited from bone marrow to outside the bone marrow are cells which can be obtained by collection of peripheral blood, or further by collection from mesenchymal tissues such as adipose, epithelial tissues such as skins, or neural tissues such as brain.

In addition, these cells also have the characteristics of having a potential to differentiate into, for example, epithelial tissues such as keratinocytes constituting the skins, or tissues of the nervous system constituting the brain, when the cells are administered to a damaged site of live bodies immediately after collection or after once being adhered onto a culture dish.

The bone marrow mesenchymal stem cells, the bone marrow mesenchymal stromal cells, the bone marrow pluripotent stromal cells, and the bone marrow pluripotent stem cells, or these cells that are recruited from the bone marrow to outside the bone marrow preferably have a potential to differentiate into osteoblasts (which can be specified by observing calcium deposition when inducing differentiation), chondrocytes (which can be specified by Alcian blue staining-positive, safranin-O staining positive, etc.), adipocytes (which can be specified by Sudan III staining positive), and, for example, mesenchymal cells such as fibroblasts, smooth muscle cells, skeletal muscle cells, stromal cells, and tendon cells; neural cells, pigment cells, epidermal cells, hair follicle cells (expressing cytokeratin family, hair keratin family, etc.), epithelial cells (e.g., epidermal keratinocytes and intestinal epithelial cells expressing cytokeratin family), and endothelial cells, and further have a potential to differentiate into cells of parenchymatous organs such as liver, kidneys, and pancreas, but the differentiated cells are not limited to the above cells.

Human bone marrow cells and human bone marrow-derived cells include, but are not limited to, cells that can be obtained by collection from bone marrow (collection of bone marrow cells), peripheral blood collection, or collection of adipose, and can be obtained as adherent cells by direct culture or by culturing an isolated monocyte fraction. Markers for the human bone marrow cells or the human bone marrow-derived cells (e.g., bone marrow mesenchymal stem cells) include all or part(s) of PDGFRα-positive, PDGFRβ-positive, Lin-negative, CD45-negative, CD44-positive, CD90-positive, CD29-positive, Flk-1-negative, CD105-positive, CD73-positive, CD90-positive, CD71-positive, Stro-1-positive, CD106-positive, CD166-positive, CD31-negative, CD271-positive, and CD11 b-negative, but not limited thereto.

In addition, murine bone marrow cells and murine bone marrow-derived cells include, but are not limited to, cells that can be obtained by collection from bone marrow (collection of bone marrow cells), peripheral blood collection, or collection of adipose, and can be obtained as adherent cells by direct culture or by culturing an isolated monocyte fraction. Markers for the murine bone marrow cells or murine marrow-derived cells (e.g., bone marrow mesenchymal stem cells) include all or part(s) of CD44-positive, PDGFRα-positive, PDGFRβ-positive, CD45-negative, Lin-negative, Sca-1-positive, c-kit-negative, CD90-positive, CD29-positive, Flk-1-negative, CD271-positive, and CD11b-negative, but not limited thereto.

In the present invention, the cells that are stimulated to migrate or the cells that are recruited from bone marrow to peripheral blood include PDGFRα-positive cells, but not limited thereto. In addition, the markers other than PDGFRα include all or part(s) of CD29-positive, CD44-positive, CD90-positive, CD271-positive, CD11b-negative, and Flk-1-negative, but not limited thereto. The PDGFRα-positive cells include, but are not limited to, PDGFRα-positive bone marrow-derived cells, PDGFRα-positive bone marrow-derived mesenchymal stem cells, tissue cells residing in the PDGFRα-positive tissues (including, e.g., fibroblasts, etc.), PDGFRα-positive bone marrow-derived cells obtained as adherent cells by cell culture of a monocyte fraction in blood obtained by collection from bone marrow (collection of bone marrow cells) or by peripheral blood collection, or the like.

The composition of the present invention may contain a substance other than at least one member of the peptides as defined in any one of the above (a) to (c). In the composition of the present invention, the substance other than at least one member of the peptides as defined in the above (a) to (c) is not particularly limited so long as the substance does not inhibit the activity of stimulating cell migration, the activity of recruiting the cells, or the activity of promoting tissue regeneration. For example, the composition of the present invention may contain, in addition to at least one member of the peptides as defined in the above (a) to (c), related molecule(s) enhancing the function(s) of the peptides as defined in the above (a) to (c), molecule(s) suppressing the actions other than the expected effects of the peptides as defined in the above (a) to (c), factors controlling proliferation or differentiation of cells, and other factors enhancing and maintaining the functions of these factors or cells.

The human HMGB1 protein in the present invention means a protein consisting of the amino acid sequence shown in SEQ ID NO: 401 as a human-derived HMGB1 protein. In addition, a peptide consisting of an amino acid sequence of position 1 to position 44 of the HMGB1 protein means a peptide consisting of the amino acid sequence shown in SEQ ID NO: 402.

The activity of stimulating cell migration of the peptide can be confirmed by, for example, a method described in Examples, or a method disclosed in WO 2012/147470, but not limited thereto.

The stability of the peptide in plasma can be confirmed by, for example, a method described in Examples, but not limited thereto.

The peptide of the present invention can be exemplified by the peptides consisting of the following amino acid sequences, but not limited to the followings.

(SEQ ID NO: 1)
(1) MGKGDPKKPR GKMSSYAFFV QTCREEHKKK HPDASVNFSE
FSKP
(SEQ ID NO: 2)
(2) MGKGDPKKPR GKMSSYAFFV QTCREEHKKK HPDASVNFSE
FSPK
(SEQ ID NO: 3)
(3) MGKGDPKKPR GKMSSYAFFV QTCREEHKKK HPDASVNFSE
FSPP
(SEQ ID NO: 4)
(4) MGKGDPKKPR GKMSSYAFFV QTCREEHKKK HPDASVNFSE
FSKA
(SEQ ID NO: 5)
(5) MGKGDPKKPR GKMSSYAFFV QTCREEHKKK HPDASVNFSE
FSKE
(SEQ ID NO: 6)
(6) MGKGDPKKPR GKMSSYAFFV QTCREEHKKK HPDASVNFSE
FSAA
(SEQ ID NO: 7)
(7) MGKGDPKKPR GKMSSYAFFV QTCREEHKKK HPDASVNFSE
FSAC
(SEQ ID NO: 8)
(8) MGKGDPKKPR GKMSSYAFFV QTCREEHKKK HPDASVNFSE
FSAD
(SEQ ID NO: 9)
(9) MGKGDPKKPR GKMSSYAFFV QTCREEHKKK HPDASVNFSE
FSAE
(SEQ ID NO: 10)
(10) MGKGDPKKPR GKMSSYAFFV QTCREEHKKK HPDASVNFSE
FSAF
(SEQ ID NO: 11)
(11) MGKGDPKKPR GKMSSYAFFV QTCREEHKKK HPDASVNFSE
FSAG
(SEQ ID NO: 12)
(12) MGKGDPKKPR GKMSSYAFFV QTCREEHKKK HPDASVNFSE
FSAH
(SEQ ID NO: 13)
(13) MGKGDPKKPR GKMSSYAFFV QTCREEHKKK HPDASVNFSE
FSAI
(SEQ ID NO: 14)
(14) MGKGDPKKPR GKMSSYAFFV QTCREEHKKK HPDASVNFSE
FSAK
(SEQ ID NO: 15)
(15) MGKGDPKKPR GKMSSYAFFV QTCREEHKKK HPDASVNFSE
FSAL
(SEQ ID NO: 16)
(16) MGKGDPKKPR GKMSSYAFFV QTCREEHKKK HPDASVNFSE
FSAM
(SEQ ID NO: 17)
(17) MGKGDPKKPR GKMSSYAFFV QTCREEHKKK HPDASVNFSE
FSAN
(SEQ ID NO: 18)
(18) MGKGDPKKPR GKMSSYAFFV QTCREEHKKK HPDASVNFSE
FSAP
(SEQ ID NO: 19)
(19) MGKGDPKKPR GKMSSYAFFV QTCREEHKKK HPDASVNFSE
FSAQ
(SEQ ID NO: 20)
(20) MGKGDPKKPR GKMSSYAFFV QTCREEHKKK HPDASVNFSE
FSAR
(SEQ ID NO: 21)
(21) MGKGDPKKPR GKMSSYAFFV QTCREEHKKK HPDASVNFSE
FSAS
(SEQ ID NO: 22)
(22) MGKGDPKKPR GKMSSYAFFV QTCREEHKKK HPDASVNFSE
FSAT
(SEQ ID NO: 23)
(23) MGKGDPKKPR GKMSSYAFFV QTCREEHKKK HPDASVNFSE
FSAV
(SEQ ID NO: 24)
(24) MGKGDPKKPR GKMSSYAFFV QTCREEHKKK HPDASVNFSE
FSAW
(SEQ ID NO: 25)
(25) MGKGDPKKPR GKMSSYAFFV QTCREEHKKK HPDASVNFSE
FSAY
(SEQ ID NO: 26)
(26) MGKGDPKKPR GKMSSYAFFV QTCREEHKKK HPDASVNFSE
FSCA
(SEQ ID NO: 27)
(27) MGKGDPKKPR GKMSSYAFFV QTCREEHKKK HPDASVNFSE
FSCC
(SEQ ID NO: 28)
(28) MGKGDPKKPR GKMSSYAFFV QTCREEHKKK HPDASVNFSE
FSCD
(SEQ ID NO: 29)
(29) MGKGDPKKPR GKMSSYAFFV QTCREEHKKK HPDASVNFSE
FSCE
(SEQ ID NO: 30)
(30) MGKGDPKKPR GKMSSYAFFV QTCREEHKKK HPDASVNFSE
FSCF
(SEQ ID NO: 31)
(31) MGKGDPKKPR GKMSSYAFFV QTCREEHKKK HPDASVNFSE
FSCG
(SEQ ID NO: 32)
(32) MGKGDPKKPR GKMSSYAFFV QTCREEHKKK HPDASVNFSE
FSCH
(SEQ ID NO: 33)
(33) MGKGDPKKPR GKMSSYAFFV QTCREEHKKK HPDASVNFSE
FSCI
(SEQ ID NO: 34)
(34) MGKGDPKKPR GKMSSYAFFV QTCREEHKKK HPDASVNFSE
FSCK
(SEQ ID NO: 35)
(35) MGKGDPKKPR GKMSSYAFFV QTCREEHKKK HPDASVNFSE
FSCL
(SEQ ID NO: 36)
(36) MGKGDPKKPR GKMSSYAFFV QTCREEHKKK HPDASVNFSE
FSCM
(SEQ ID NO: 37)
(37) MGKGDPKKPR GKMSSYAFFV QTCREEHKKK HPDASVNFSE
FSCN
(SEQ ID NO: 38)
(38) MGKGDPKKPR GKMSSYAFFV QTCREEHKKK HPDASVNFSE
FSCP
(SEQ ID NO: 39)
(39) MGKGDPKKPR GKMSSYAFFV QTCREEHKKK HPDASVNFSE
FSCQ
(SEQ ID NO: 40)
(40) MGKGDPKKPR GKMSSYAFFV QTCREEHKKK HPDASVNFSE
FSCR
(SEQ ID NO: 41)
(41) MGKGDPKKPR GKMSSYAFFV QTCREEHKKK HPDASVNFSE
FSCS
(SEQ ID NO: 42)
(42) MGKGDPKKPR GKMSSYAFFV QTCREEHKKK HPDASVNFSE
FSCT
(SEQ ID NO: 43)
(43) MGKGDPKKPR GKMSSYAFFV QTCREEHKKK HPDASVNFSE
FSCV
(SEQ ID NO: 44)
(44) MGKGDPKKPR GKMSSYAFFV QTCREEHKKK HPDASVNFSE
FSCW
(SEQ ID NO: 45)
(45) MGKGDPKKPR GKMSSYAFFV QTCREEHKKK HPDASVNFSE
FSCY
(SEQ ID NO: 46)
(46) MGKGDPKKPR GKMSSYAFFV QTCREEHKKK HPDASVNFSE
FSDA
(SEQ ID NO: 47)
(47) MGKGDPKKPR GKMSSYAFFV QTCREEHKKK HPDASVNFSE
FSDC
(SEQ ID NO: 48)
(48) MGKGDPKKPR GKMSSYAFFV QTCREEHKKK HPDASVNFSE
FSDD
(SEQ ID NO: 49)
(49) MGKGDPKKPR GKMSSYAFFV QTCREEHKKK HPDASVNFSE
FSDE
(SEQ ID NO: 50)
(50) MGKGDPKKPR GKMSSYAFFV QTCREEHKKK HPDASVNFSE
FSDF
(SEQ ID NO: 51)
(51) MGKGDPKKPR GKMSSYAFFV QTCREEHKKK
HPDASVNFSE FSDG
(SEQ ID NO: 52)
(52) MGKGDPKKPR GKMSSYAFFV QTCREEHKKK HPDASVNFSE
FSDH
(SEQ ID NO: 53)
(53) MGKGDPKKPR GKMSSYAFFV QTCREEHKKK HPDASVNFSE
FSDI
(SEQ ID NO: 54)
(54) MGKGDPKKPR GKMSSYAFFV QTCREEHKKK HPDASVNFSE
FSDK
(SEQ ID NO: 55)
(55) MGKGDPKKPR GKMSSYAFFV QTCREEHKKK HPDASVNFSE
FSDL
(SEQ ID NO: 56)
(56) MGKGDPKKPR GKMSSYAFFV QTCREEHKKK HPDASVNFSE
FSDM
(SEQ ID NO: 57)
(57) MGKGDPKKPR GKMSSYAFFV QTCREEHKKK HPDASVNFSE
FSDN
(SEQ ID NO: 58)
(58) MGKGDPKKPR GKMSSYAFFV QTCREEHKKK HPDASVNFSE
FSDP
(SEQ ID NO: 59)
(59) MGKGDPKKPR GKMSSYAFFV QTCREEHKKK HPDASVNFSE
FSDQ
(SEQ ID NO: 60)
(60) MGKGDPKKPR GKMSSYAFFV QTCREEHKKK HPDASVNFSE
FSDR
(SEQ ID NO: 61)
(61) MGKGDPKKPR GKMSSYAFFV QTCREEHKKK HPDASVNFSE
FSDS
(SEQ ID NO: 62)
(62) MGKGDPKKPR GKMSSYAFFV QTCREEHKKK HPDASVNFSE
FSDT
(SEQ ID NO: 63)
(63) MGKGDPKKPR GKMSSYAFFV QTCREEHKKK HPDASVNFSE
FSDV
(SEQ ID NO: 64)
(64) MGKGDPKKPR GKMSSYAFFV QTCREEHKKK HPDASVNFSE
FSDW
(SEQ ID NO: 65)
(65) MGKGDPKKPR GKMSSYAFFV QTCREEHKKK HPDASVNFSE
FSDY
(SEQ ID NO: 66)
(66) MGKGDPKKPR GKMSSYAFFV QTCREEHKKK HPDASVNFSE
FSEA
(SEQ ID NO: 67)
(67) MGKGDPKKPR GKMSSYAFFV QTCREEHKKK HPDASVNFSE
FSEC
(SEQ ID NO: 68)
(68) MGKGDPKKPR GKMSSYAFFV QTCREEHKKK HPDASVNFSE
FSED
(SEQ ID NO: 69)
(69) MGKGDPKKPR GKMSSYAFFV QTCREEHKKK HPDASVNFSE
FSEE
(SEQ ID NO: 70)
(70) MGKGDPKKPR GKMSSYAFFV QTCREEHKKK HPDASVNFSE
FSEF
(SEQ ID NO: 71)
(71) MGKGDPKKPR GKMSSYAFFV QTCREEHKKK HPDASVNFSE
FSEG
(SEQ ID NO: 72)
(72) MGKGDPKKPR GKMSSYAFFV QTCREEHKKK HPDASVNFSE
FSEH
(SEQ ID NO: 73)
(73) MGKGDPKKPR GKMSSYAFFV QTCREEHKKK HPDASVNFSE
FSEI
(SEQ ID NO: 74)
(74) MGKGDPKKPR GKMSSYAFFV QTCREEHKKK HPDASVNFSE
FSEK
(SEQ ID NO: 75)
(75) MGKGDPKKPR GKMSSYAFFV QTCREEHKKK HPDASVNFSE
FSEL
(SEQ ID NO: 76)
(76) MGKGDPKKPR GKMSSYAFFV QTCREEHKKK HPDASVNFSE
FSEM
(SEQ ID NO: 77)
(77) MGKGDPKKPR GKMSSYAFFV QTCREEHKKK HPDASVNFSE
FSEN
(SEQ ID NO: 78)
(78) MGKGDPKKPR GKMSSYAFFV QTCREEHKKK HPDASVNFSE
FSEP
(SEQ ID NO: 79)
(79) MGKGDPKKPR GKMSSYAFFV QTCREEHKKK HPDASVNFSE
FSEQ
(SEQ ID NO: 80)
(80) MGKGDPKKPR GKMSSYAFFV QTCREEHKKK HPDASVNFSE
FSER
(SEQ ID NO: 81)
(81) MGKGDPKKPR GKMSSYAFFV QTCREEHKKK HPDASVNFSE
FSES
(SEQ ID NO: 82)
(82) MGKGDPKKPR GKMSSYAFFV QTCREEHKKK HPDASVNFSE
FSET
(SEQ ID NO: 83)
(83) MGKGDPKKPR GKMSSYAFFV QTCREEHKKK HPDASVNFSE
FSEV
(SEQ ID NO: 84)
(84) MGKGDPKKPR GKMSSYAFFV QTCREEHKKK HPDASVNFSE
FSEW
(SEQ ID NO: 85)
(85) MGKGDPKKPR GKMSSYAFFV QTCREEHKKK HPDASVNFSE
FSEY
(SEQ ID NO: 86)
(86) MGKGDPKKPR GKMSSYAFFV QTCREEHKKK HPDASVNFSE
FSFA
(SEQ ID NO: 87)
(87) MGKGDPKKPR GKMSSYAFFV QTCREEHKKK HPDASVNFSE
FSFC
(SEQ ID NO: 88)
(88) MGKGDPKKPR GKMSSYAFFV QTCREEHKKK HPDASVNFSE
FSFD
(SEQ ID NO: 89)
(89) MGKGDPKKPR GKMSSYAFFV QTCREEHKKK HPDASVNFSE
FSFE
(SEQ ID NO: 90)
(90) MGKGDPKKPR GKMSSYAFFV QTCREEHKKK HPDASVNFSE
FSFF
(SEQ ID NO: 91)
(91) MGKGDPKKPR GKMSSYAFFV QTCREEHKKK HPDASVNFSE
FSFG
(SEQ ID NO: 92)
(92) MGKGDPKKPR GKMSSYAFFV QTCREEHKKK HPDASVNFSE
FSFH
(SEQ ID NO: 93)
(93) MGKGDPKKPR GKMSSYAFFV QTCREEHKKK HPDASVNFSE
FSFI
(SEQ ID NO: 94)
(94) MGKGDPKKPR GKMSSYAFFV QTCREEHKKK HPDASVNFSE
FSFK
(SEQ ID NO: 95)
(95) MGKGDPKKPR GKMSSYAFFV QTCREEHKKK HPDASVNFSE
FSFL
(SEQ ID NO: 96)
(96) MGKGDPKKPR GKMSSYAFFV QTCREEHKKK HPDASVNFSE
FSFM
(SEQ ID NO: 97)
(97) MGKGDPKKPR GKMSSYAFFV QTCREEHKKK HPDASVNFSE
FSFN
(SEQ ID NO: 98)
(98) MGKGDPKKPR GKMSSYAFFV QTCREEHKKK HPDASVNFSE
FSFP
(SEQ ID NO: 99)
(99) MGKGDPKKPR GKMSSYAFFV QTCREEHKKK HPDASVNFSE
FSFQ
(SEQ ID NO: 100)
(100) MGKGDPKKPR GKMSSYAFFV QTCREEHKKK
HPDASVNFSE FSFR
(SEQ ID NO: 101)
(101) MGKGDPKKPR GKMSSYAFFV QTCREEHKKK
HPDASVNFSE FSFS
(SEQ ID NO: 102)
(102) MGKGDPKKPR GKMSSYAFFV QTCREEHKKK
HPDASVNFSE FSFT
(SEQ ID NO: 103)
(103) MGKGDPKKPR GKMSSYAFFV QTCREEHKKK
HPDASVNFSE FSFV
(SEQ ID NO: 104)
(104) MGKGDPKKPR GKMSSYAFFV QTCREEHKKK
HPDASVNFSE FSFW
(SEQ ID NO: 105)
(105) MGKGDPKKPR GKMSSYAFFV QTCREEHKKK
HPDASVNFSE FSFY
(SEQ ID NO: 106)
(106) MGKGDPKKPR GKMSSYAFFV QTCREEHKKK
HPDASVNFSE FSGA
(SEQ ID NO: 107)
(107) MGKGDPKKPR GKMSSYAFFV QTCREEHKKK
HPDASVNFSE FSGC
(SEQ ID NO: 108)
(108) MGKGDPKKPR GKMSSYAFFV QTCREEHKKK
HPDASVNFSE FSGD
(SEQ ID NO: 109)
(109) MGKGDPKKPR GKMSSYAFFV QTCREEHKKK
HPDASVNFSE FSGE
(SEQ ID NO: 110)
(110) MGKGDPKKPR GKMSSYAFFV QTCREEHKKK
HPDASVNFSE FSGF
(SEQ ID NO: 111)
(111) MGKGDPKKPR GKMSSYAFFV QTCREEHKKK
HPDASVNFSE FSGG
(SEQ ID NO: 112)
(112) MGKGDPKKPR GKMSSYAFFV QTCREEHKKK
HPDASVNFSE FSGH
(SEQ ID NO: 113)
(113) MGKGDPKKPR GKMSSYAFFV QTCREEHKKK
HPDASVNFSE FSGI
(SEQ ID NO: 114)
(114) MGKGDPKKPR GKMSSYAFFV QTCREEHKKK
HPDASVNFSE FSGK
(SEQ ID NO: 115)
(115) MGKGDPKKPR GKMSSYAFFV QTCREEHKKK
HPDASVNFSE FSGL
(SEQ ID NO: 116)
(116) MGKGDPKKPR GKMSSYAFFV QTCREEHKKK
HPDASVNFSE FSGM
(SEQ ID NO: 117)
(117) MGKGDPKKPR GKMSSYAFFV QTCREEHKKK
HPDASVNFSE FSGN
(SEQ ID NO: 118)
(118) MGKGDPKKPR GKMSSYAFFV QTCREEHKKK
HPDASVNFSE FSGP
(SEQ ID NO: 119)
(119) MGKGDPKKPR GKMSSYAFFV QTCREEHKKK
HPDASVNFSE FSGQ
(SEQ ID NO: 120)
(120) MGKGDPKKPR GKMSSYAFFV QTCREEHKKK
HPDASVNFSE FSGR
(SEQ ID NO: 121)
(121) MGKGDPKKPR GKMSSYAFFV QTCREEHKKK
HPDASVNFSE FSGS
(SEQ ID NO: 122)
(122) MGKGDPKKPR GKMSSYAFFV QTCREEHKKK
HPDASVNFSE FSGT
(SEQ ID NO: 123)
(123) MGKGDPKKPR GKMSSYAFFV QTCREEHKKK
HPDASVNFSE FSGV
(SEQ ID NO: 124)
(124) MGKGDPKKPR GKMSSYAFFV QTCREEHKKK
HPDASVNFSE FSGW
(SEQ ID NO: 125)
(125) MGKGDPKKPR GKMSSYAFFV QTCREEHKKK
HPDASVNFSE FSGY
(SEQ ID NO: 126)
(126) MGKGDPKKPR GKMSSYAFFV QTCREEHKKK
HPDASVNFSE FSHA
(SEQ ID NO: 127)
(127) MGKGDPKKPR GKMSSYAFFV QTCREEHKKK
HPDASVNFSE FSHC
(SEQ ID NO: 128)
(128) MGKGDPKKPR GKMSSYAFFV QTCREEHKKK
HPDASVNFSE FSHD
(SEQ ID NO: 129)
(129) MGKGDPKKPR GKMSSYAFFV QTCREEHKKK
HPDASVNFSE FSHE
(SEQ ID NO: 130)
(130) MGKGDPKKPR GKMSSYAFFV QTCREEHKKK
HPDASVNFSE FSHF
(SEQ ID NO: 131)
(131) MGKGDPKKPR GKMSSYAFFV QTCREEHKKK
HPDASVNFSE FSHG
(SEQ ID NO: 132)
(132) MGKGDPKKPR GKMSSYAFFV QTCREEHKKK
HPDASVNFSE FSHH
(SEQ ID NO: 133)
(133) MGKGDPKKPR GKMSSYAFFV QTCREEHKKK
HPDASVNFSE FSHI
(SEQ ID NO: 134)
(134) MGKGDPKKPR GKMSSYAFFV QTCREEHKKK
HPDASVNFSE FSHK
(SEQ ID NO: 135)
(135) MGKGDPKKPR GKMSSYAFFV QTCREEHKKK
HPDASVNFSE FSHL
(SEQ ID NO: 136)
(136) MGKGDPKKPR GKMSSYAFFV QTCREEHKKK
HPDASVNFSE FSHM
(SEQ ID NO: 137)
(137) MGKGDPKKPR GKMSSYAFFV QTCREEHKKK
HPDASVNFSE FSHN
(SEQ ID NO: 138)
(138) MGKGDPKKPR GKMSSYAFFV QTCREEHKKK
HPDASVNFSE FSHP
(SEQ ID NO: 139)
(139) MGKGDPKKPR GKMSSYAFFV QTCREEHKKK
HPDASVNFSE FSHQ
(SEQ ID NO: 140)
(140) MGKGDPKKPR GKMSSYAFFV QTCREEHKKK
HPDASVNFSE FSHR
(SEQ ID NO: 141)
(141) MGKGDPKKPR GKMSSYAFFV QTCREEHKKK
HPDASVNFSE FSHS
(SEQ ID NO: 142)
(142) MGKGDPKKPR GKMSSYAFFV QTCREEHKKK
HPDASVNFSE FSHT
(SEQ ID NO: 143)
(143) MGKGDPKKPR GKMSSYAFFV QTCREEHKKK
HPDASVNFSE FSHV
(SEQ ID NO: 144)
(144) MGKGDPKKPR GKMSSYAFFV QTCREEHKKK
HPDASVNFSE FSHW
(SEQ ID NO: 145)
(145) MGKGDPKKPR GKMSSYAFFV QTCREEHKKK
HPDASVNFSE FSHY
(SEQ ID NO: 146)
(146) MGKGDPKKPR GKMSSYAFFV QTCREEHKKK
HPDASVNFSE FSIA
(SEQ ID NO: 147)
(147) MGKGDPKKPR GKMSSYAFFV QTCREEHKKK
HPDASVNFSE FSIC
(SEQ ID NO: 148)
(148) MGKGDPKKPR GKMSSYAFFV QTCREEHKKK
HPDASVNFSE FSID
(SEQ ID NO: 149)
(149) MGKGDPKKPR GKMSSYAFFV QTCREEHKKK
HPDASVNFSE FSIE
(SEQ ID NO: 150)
(150) MGKGDPKKPR GKMSSYAFFV QTCREEHKKK
HPDASVNFSE FSIF
(SEQ ID NO: 151)
(151) MGKGDPKKPR GKMSSYAFFV QTCREEHKKK
HPDASVNFSE FSIG
(SEQ ID NO: 152)
(152) MGKGDPKKPR GKMSSYAFFV QTCREEHKKK
HPDASVNFSE FSIH
(SEQ ID NO: 153)
(153) MGKGDPKKPR GKMSSYAFFV QTCREEHKKK
HPDASVNFSE FSII
(SEQ ID NO: 154)
(154) MGKGDPKKPR GKMSSYAFFV QTCREEHKKK
HPDASVNFSE FSIK
(SEQ ID NO: 155)
(155) MGKGDPKKPR GKMSSYAFFV QTCREEHKKK
HPDASVNFSE FSIL
(SEQ ID NO: 156)
(156) MGKGDPKKPR GKMSSYAFFV QTCREEHKKK
HPDASVNFSE FSIM
(SEQ ID NO: 157)
(157) MGKGDPKKPR GKMSSYAFFV QTCREEHKKK
HPDASVNFSE FSIN
(SEQ ID NO: 158)
(158) MGKGDPKKPR GKMSSYAFFV QTCREEHKKK
HPDASVNFSE FSIP
(SEQ ID NO: 159)
(159) MGKGDPKKPR GKMSSYAFFV QTCREEHKKK
HPDASVNFSE FSIQ
(SEQ ID NO: 160)
(160) MGKGDPKKPR GKMSSYAFFV QTCREEHKKK
HPDASVNFSE FSIR
(SEQ ID NO: 161)
(161) MGKGDPKKPR GKMSSYAFFV QTCREEHKKK
HPDASVNFSE FSIS
(SEQ ID NO: 162)
(162) MGKGDPKKPR GKMSSYAFFV QTCREEHKKK
HPDASVNFSE FSIT
(SEQ ID NO: 163)
(163) MGKGDPKKPR GKMSSYAFFV QTCREEHKKK
HPDASVNFSE FSIV
(SEQ ID NO: 164)
(164) MGKGDPKKPR GKMSSYAFFV QTCREEHKKK
HPDASVNFSE FSIW
(SEQ ID NO: 165)
(165) MGKGDPKKPR GKMSSYAFFV QTCREEHKKK
HPDASVNFSE FSIY
(SEQ ID NO: 166)
(166) MGKGDPKKPR GKMSSYAFFV QTCREEHKKK
HPDASVNFSE FSKC
(SEQ ID NO: 167)
(167) MGKGDPKKPR GKMSSYAFFV QTCREEHKKK
HPDASVNFSE FSKD
(SEQ ID NO: 168)
(168) MGKGDPKKPR GKMSSYAFFV QTCREEHKKK
HPDASVNFSE FSKF
(SEQ ID NO: 169)
(169) MGKGDPKKPR GKMSSYAFFV QTCREEHKKK
HPDASVNFSE FSKG
(SEQ ID NO: 170)
(170) MGKGDPKKPR GKMSSYAFFV QTCREEHKKK
HPDASVNFSE FSKH
(SEQ ID NO: 171)
(171) MGKGDPKKPR GKMSSYAFFV QTCREEHKKK
HPDASVNFSE FSKI
(SEQ ID NO: 172)
(172) MGKGDPKKPR GKMSSYAFFV QTCREEHKKK
HPDASVNFSE FSKL
(SEQ ID NO: 173)
(173) MGKGDPKKPR GKMSSYAFFV QTCREEHKKK
HPDASVNFSE FSKM
(SEQ ID NO: 174)
(174) MGKGDPKKPR GKMSSYAFFV QTCREEHKKK
HPDASVNFSE FSKN
(SEQ ID NO: 175)
(175) MGKGDPKKPR GKMSSYAFFV QTCREEHKKK
HPDASVNFSE FSKQ
(SEQ ID NO: 176)
(176) MGKGDPKKPR GKMSSYAFFV QTCREEHKKK
HPDASVNFSE FSKR
(SEQ ID NO: 177)
(177) MGKGDPKKPR GKMSSYAFFV QTCREEHKKK
HPDASVNFSE FSKS
(SEQ ID NO: 178)
(178) MGKGDPKKPR GKMSSYAFFV QTCREEHKKK
HPDASVNFSE FSKT
(SEQ ID NO: 179)
(179) MGKGDPKKPR GKMSSYAFFV QTCREEHKKK
HPDASVNFSE FSKV
(SEQ ID NO: 180)
(180) MGKGDPKKPR GKMSSYAFFV QTCREEHKKK
HPDASVNFSE FSKW
(SEQ ID NO: 181)
(181) MGKGDPKKPR GKMSSYAFFV QTCREEHKKK
HPDASVNFSE FSKY
(SEQ ID NO: 182)
(182) MGKGDPKKPR GKMSSYAFFV QTCREEHKKK
HPDASVNFSE FSLA
(SEQ ID NO: 183)
(183) MGKGDPKKPR GKMSSYAFFV QTCREEHKKK
HPDASVNFSE FSLC
(SEQ ID NO: 184)
(184) MGKGDPKKPR GKMSSYAFFV QTCREEHKKK
HPDASVNFSE FSLD
(SEQ ID NO: 185)
(185) MGKGDPKKPR GKMSSYAFFV QTCREEHKKK
HPDASVNFSE FSLE
(SEQ ID NO: 186)
(186) MGKGDPKKPR GKMSSYAFFV QTCREEHKKK
HPDASVNFSE FSLF
(SEQ ID NO: 187)
(187) MGKGDPKKPR GKMSSYAFFV QTCREEHKKK
HPDASVNFSE FSLG
(SEQ ID NO: 188)
(188) MGKGDPKKPR GKMSSYAFFV QTCREEHKKK
HPDASVNFSE FSLH
(SEQ ID NO: 189)
(189) MGKGDPKKPR GKMSSYAFFV QTCREEHKKK
HPDASVNFSE FSLI
(SEQ ID NO: 190)
(190) MGKGDPKKPR GKMSSYAFFV QTCREEHKKK
HPDASVNFSE FSLK
(SEQ ID NO: 191)
(191) MGKGDPKKPR GKMSSYAFFV QTCREEHKKK
HPDASVNFSE FSLL
(SEQ ID NO: 192)
(192) MGKGDPKKPR GKMSSYAFFV QTCREEHKKK
HPDASVNFSE FSLM
(SEQ ID NO: 193)
(193) MGKGDPKKPR GKMSSYAFFV QTCREEHKKK
HPDASVNFSE FSLN
(SEQ ID NO: 194)
(194) MGKGDPKKPR GKMSSYAFFV QTCREEHKKK
HPDASVNFSE FSLP
(SEQ ID NO: 195)
(195) MGKGDPKKPR GKMSSYAFFV QTCREEHKKK
HPDASVNFSE FSLQ
(SEQ ID NO: 196)
(196) MGKGDPKKPR GKMSSYAFFV QTCREEHKKK
HPDASVNFSE FSLR
(SEQ ID NO: 197)
(197) MGKGDPKKPR GKMSSYAFFV QTCREEHKKK
HPDASVNFSE FSLS
(SEQ ID NO: 198)
(198) MGKGDPKKPR GKMSSYAFFV QTCREEHKKK
HPDASVNFSE FSLT
(SEQ ID NO: 199)
(199) MGKGDPKKPR GKMSSYAFFV QTCREEHKKK
HPDASVNFSE FSLV
(SEQ ID NO: 200)
(200) MGKGDPKKPR GKMSSYAFFV QTCREEHKKK
HPDASVNFSE FSLW
(SEQ ID NO: 201)
(201) MGKGDPKKPR GKMSSYAFFV QTCREEHKKK
HPDASVNFSE FSLY
(SEQ ID NO: 202)
(202) MGKGDPKKPR GKMSSYAFFV QTCREEHKKK
HPDASVNFSE FSMA
(SEQ ID NO: 203)
(203) MGKGDPKKPR GKMSSYAFFV QTCREEHKKK
HPDASVNFSE FSMC
(SEQ ID NO: 204)
(204) MGKGDPKKPR GKMSSYAFFV QTCREEHKKK
HPDASVNFSE FSMD
(SEQ ID NO: 205)
(205) MGKGDPKKPR GKMSSYAFFV QTCREEHKKK
HPDASVNFSE FSME
(SEQ ID NO: 206)
(206) MGKGDPKKPR GKMSSYAFFV QTCREEHKKK
HPDASVNFSE FSMF
(SEQ ID NO: 207)
(207) MGKGDPKKPR GKMSSYAFFV QTCREEHKKK
HPDASVNFSE FSMG
(SEQ ID NO: 208)
(208) MGKGDPKKPR GKMSSYAFFV QTCREEHKKK
HPDASVNFSE FSMH
(SEQ ID NO: 209)
(209) MGKGDPKKPR GKMSSYAFFV QTCREEHKKK
HPDASVNFSE FSMI
(SEQ ID NO: 210)
(210) MGKGDPKKPR GKMSSYAFFV QTCREEHKKK
HPDASVNFSE FSMK
(SEQ ID NO: 211)
(211) MGKGDPKKPR GKMSSYAFFV QTCREEHKKK
HPDASVNFSE FSML
(SEQ ID NO: 212)
(212) MGKGDPKKPR GKMSSYAFFV QTCREEHKKK
HPDASVNFSE FSMM
(SEQ ID NO: 213)
(213) MGKGDPKKPR GKMSSYAFFV QTCREEHKKK
HPDASVNFSE FSMN
(SEQ ID NO: 214)
(214) MGKGDPKKPR GKMSSYAFFV QTCREEHKKK
HPDASVNFSE FSMP
(SEQ ID NO: 215)
(215) MGKGDPKKPR GKMSSYAFFV QTCREEHKKK
HPDASVNFSE FSMQ
(SEQ ID NO: 216)
(216) MGKGDPKKPR GKMSSYAFFV QTCREEHKKK
HPDASVNFSE FSMR
(SEQ ID NO: 217)
(217) MGKGDPKKPR GKMSSYAFFV QTCREEHKKK
HPDASVNFSE FSMS
(SEQ ID NO: 218)
(218) MGKGDPKKPR GKMSSYAFFV QTCREEHKKK
HPDASVNFSE FSMT
(SEQ ID NO: 219)
(219) MGKGDPKKPR GKMSSYAFFV QTCREEHKKK
HPDASVNFSE FSMV
(SEQ ID NO: 220)
(220) MGKGDPKKPR GKMSSYAFFV QTCREEHKKK
HPDASVNFSE FSMW
(SEQ ID NO: 221)
(221) MGKGDPKKPR GKMSSYAFFV QTCREEHKKK
HPDASVNFSE FSMY
(SEQ ID NO: 222)
(222) MGKGDPKKPR GKMSSYAFFV QTCREEHKKK
HPDASVNFSE FSNA
(SEQ ID NO: 223)
(223) MGKGDPKKPR GKMSSYAFFV QTCREEHKKK
HPDASVNFSE FSNC
(SEQ ID NO: 224)
(224) MGKGDPKKPR GKMSSYAFFV QTCREEHKKK
HPDASVNFSE FSND
(SEQ ID NO: 225)
(225) MGKGDPKKPR GKMSSYAFFV QTCREEHKKK
HPDASVNFSE FSNE
(SEQ ID NO: 226)
(226) MGKGDPKKPR GKMSSYAFFV QTCREEHKKK
HPDASVNFSE FSNF
(SEQ ID NO: 227)
(227) MGKGDPKKPR GKMSSYAFFV QTCREEHKKK
HPDASVNFSE FSNG
(SEQ ID NO: 228)
(228) MGKGDPKKPR GKMSSYAFFV QTCREEHKKK
HPDASVNFSE FSNH
(SEQ ID NO: 229)
(229) MGKGDPKKPR GKMSSYAFFV QTCREEHKKK
HPDASVNFSE FSNI
(SEQ ID NO: 230)
(230) MGKGDPKKPR GKMSSYAFFV QTCREEHKKK
HPDASVNFSE FSNK
(SEQ ID NO: 231)
(231) MGKGDPKKPR GKMSSYAFFV QTCREEHKKK
HPDASVNFSE FSNL
(SEQ ID NO: 232)
(232) MGKGDPKKPR GKMSSYAFFV QTCREEHKKK
HPDASVNFSE FSNM
(SEQ ID NO: 233)
(233) MGKGDPKKPR GKMSSYAFFV QTCREEHKKK
HPDASVNFSE FSNN
(SEQ ID NO: 234)
(234) MGKGDPKKPR GKMSSYAFFV QTCREEHKKK
HPDASVNFSE FSNP
(SEQ ID NO: 235)
(235) MGKGDPKKPR GKMSSYAFFV QTCREEHKKK
HPDASVNFSE FSNQ
(SEQ ID NO: 236)
(236) MGKGDPKKPR GKMSSYAFFV QTCREEHKKK
HPDASVNFSE FSNR
(SEQ ID NO: 237)
(237) MGKGDPKKPR GKMSSYAFFV QTCREEHKKK
HPDASVNFSE FSNS
(SEQ ID NO: 238)
(238) MGKGDPKKPR GKMSSYAFFV QTCREEHKKK
HPDASVNFSE FSNT
(SEQ ID NO: 239)
(239) MGKGDPKKPR GKMSSYAFFV QTCREEHKKK
HPDASVNFSE FSNV
(SEQ ID NO: 240)
(240) MGKGDPKKPR GKMSSYAFFV QTCREEHKKK
HPDASVNFSE FSNW
(SEQ ID NO: 241)
(241) MGKGDPKKPR GKMSSYAFFV QTCREEHKKK
HPDASVNFSE FSNY
(SEQ ID NO: 242)
(242) MGKGDPKKPR GKMSSYAFFV QTCREEHKKK
HPDASVNFSE FSPA
(SEQ ID NO: 243)
(243) MGKGDPKKPR GKMSSYAFFV QTCREEHKKK
HPDASVNFSE FSPC
(SEQ ID NO: 244)
(244) MGKGDPKKPR GKMSSYAFFV QTCREEHKKK
HPDASVNFSE FSPD
(SEQ ID NO: 245)
(245) MGKGDPKKPR GKMSSYAFFV QTCREEHKKK
HPDASVNFSE FSPE
(SEQ ID NO: 246)
(246) MGKGDPKKPR GKMSSYAFFV QTCREEHKKK
HPDASVNFSE FSPF
(SEQ ID NO: 247)
(247) MGKGDPKKPR GKMSSYAFFV QTCREEHKKK
HPDASVNFSE FSPG
(SEQ ID NO: 248)
(248) MGKGDPKKPR GKMSSYAFFV QTCREEHKKK
HPDASVNFSE FSPH
(SEQ ID NO: 249)
(249) MGKGDPKKPR GKMSSYAFFV QTCREEHKKK
HPDASVNFSE FSPI
(SEQ ID NO: 250)
(250) MGKGDPKKPR GKMSSYAFFV QTCREEHKKK
HPDASVNFSE FSPL
(SEQ ID NO: 251)
(251) MGKGDPKKPR GKMSSYAFFV QTCREEHKKK
HPDASVNFSE FSPM
(SEQ ID NO: 252)
(252) MGKGDPKKPR GKMSSYAFFV QTCREEHKKK
HPDASVNFSE FSPN
(SEQ ID NO: 253)
(253) MGKGDPKKPR GKMSSYAFFV QTCREEHKKK
HPDASVNFSE FSPQ
(SEQ ID NO: 254)
(254) MGKGDPKKPR GKMSSYAFFV QTCREEHKKK
HPDASVNFSE FSPR
(SEQ ID NO: 255)
(255) MGKGDPKKPR GKMSSYAFFV QTCREEHKKK
HPDASVNFSE FSPS
(SEQ ID NO: 256)
(256) MGKGDPKKPR GKMSSYAFFV QTCREEHKKK
HPDASVNFSE FSPT
(SEQ ID NO: 257)
(257) MGKGDPKKPR GKMSSYAFFV QTCREEHKKK
HPDASVNFSE FSPV
(SEQ ID NO: 258)
(258) MGKGDPKKPR GKMSSYAFFV QTCREEHKKK
HPDASVNFSE FSPW
(SEQ ID NO: 259)
(259) MGKGDPKKPR GKMSSYAFFV QTCREEHKKK
HPDASVNFSE FSPY
(SEQ ID NO: 260)
(260) MGKGDPKKPR GKMSSYAFFV QTCREEHKKK
HPDASVNFSE FSQA
(SEQ ID NO: 261)
(261) MGKGDPKKPR GKMSSYAFFV QTCREEHKKK
HPDASVNFSE FSQC
(SEQ ID NO: 262)
(262) MGKGDPKKPR GKMSSYAFFV QTCREEHKKK
HPDASVNFSE FSQD
(SEQ ID NO: 263)
(263) MGKGDPKKPR GKMSSYAFFV QTCREEHKKK
HPDASVNFSE FSQE
(SEQ ID NO: 264)
(264) MGKGDPKKPR GKMSSYAFFV QTCREEHKKK
HPDASVNFSE FSQF
(SEQ ID NO: 265)
(265) MGKGDPKKPR GKMSSYAFFV QTCREEHKKK
HPDASVNFSE FSQG
(SEQ ID NO: 266)
(266) MGKGDPKKPR GKMSSYAFFV QTCREEHKKK
HPDASVNFSE FSQH
(SEQ ID NO: 267)
(267) MGKGDPKKPR GKMSSYAFFV QTCREEHKKK
HPDASVNFSE FSQI
(SEQ ID NO: 268)
(268) MGKGDPKKPR GKMSSYAFFV QTCREEHKKK
HPDASVNFSE FSQK
(SEQ ID NO: 269)
(269) MGKGDPKKPR GKMSSYAFFV QTCREEHKKK
HPDASVNFSE FSQL
(SEQ ID NO: 270)
(270) MGKGDPKKPR GKMSSYAFFV QTCREEHKKK
HPDASVNFSE FSQM
(SEQ ID NO: 271)
(271) MGKGDPKKPR GKMSSYAFFV QTCREEHKKK
HPDASVNFSE FSQN
(SEQ ID NO: 272)
(272) MGKGDPKKPR GKMSSYAFFV QTCREEHKKK
HPDASVNFSE FSQP
(SEQ ID NO: 273)
(273) MGKGDPKKPR GKMSSYAFFV QTCREEHKKK
HPDASVNFSE FSQQ
(SEQ ID NO: 274)
(274) MGKGDPKKPR GKMSSYAFFV QTCREEHKKK
HPDASVNFSE FSQR
(SEQ ID NO: 275)
(275) MGKGDPKKPR GKMSSYAFFV QTCREEHKKK
HPDASVNFSE FSQS
(SEQ ID NO: 276)
(276) MGKGDPKKPR GKMSSYAFFV QTCREEHKKK
HPDASVNFSE FSQT
(SEQ ID NO: 277)
(277) MGKGDPKKPR GKMSSYAFFV QTCREEHKKK
HPDASVNFSE FSQV
(SEQ ID NO: 278)
(278) MGKGDPKKPR GKMSSYAFFV QTCREEHKKK
HPDASVNFSE FSQW
(SEQ ID NO: 279)
(279) MGKGDPKKPR GKMSSYAFFV QTCREEHKKK
HPDASVNFSE FSQY
(SEQ ID NO: 280)
(280) MGKGDPKKPR GKMSSYAFFV QTCREEHKKK
HPDASVNFSE FSRA
(SEQ ID NO: 281)
(281) MGKGDPKKPR GKMSSYAFFV QTCREEHKKK
HPDASVNFSE FSRC
(SEQ ID NO: 282)
(282) MGKGDPKKPR GKMSSYAFFV QTCREEHKKK
HPDASVNFSE FSRD
(SEQ ID NO: 283)
(283) MGKGDPKKPR GKMSSYAFFV QTCREEHKKK
HPDASVNFSE FSRE
(SEQ ID NO: 284)
(284) MGKGDPKKPR GKMSSYAFFV QTCREEHKKK
HPDASVNFSE FSRF
(SEQ ID NO: 285)
(285) MGKGDPKKPR GKMSSYAFFV QTCREEHKKK
HPDASVNFSE FSRG
(SEQ ID NO: 286)
(286) MGKGDPKKPR GKMSSYAFFV QTCREEHKKK
HPDASVNFSE FSRH
(SEQ ID NO: 287)
(287) MGKGDPKKPR GKMSSYAFFV QTCREEHKKK
HPDASVNFSE FSRI
(SEQ ID NO: 288)
(288) MGKGDPKKPR GKMSSYAFFV QTCREEHKKK
HPDASVNFSE FSRK
(SEQ ID NO: 289)
(289) MGKGDPKKPR GKMSSYAFFV QTCREEHKKK
HPDASVNFSE FSRL
(SEQ ID NO: 290)
(290) MGKGDPKKPR GKMSSYAFFV QTCREEHKKK
HPDASVNFSE FSRM
(SEQ ID NO: 291)
(291) MGKGDPKKPR GKMSSYAFFV QTCREEHKKK
HPDASVNFSE FSRN
(SEQ ID NO: 292)
(292) MGKGDPKKPR GKMSSYAFFV QTCREEHKKK
HPDASVNFSE FSRP
(SEQ ID NO: 293)
(293) MGKGDPKKPR GKMSSYAFFV QTCREEHKKK
HPDASVNFSE FSRQ
(SEQ ID NO: 294)
(294) MGKGDPKKPR GKMSSYAFFV QTCREEHKKK
HPDASVNFSE FSRR
(SEQ ID NO: 295)
(295) MGKGDPKKPR GKMSSYAFFV QTCREEHKKK
HPDASVNFSE FSRS
(SEQ ID NO: 296)
(296) MGKGDPKKPR GKMSSYAFFV QTCREEHKKK
HPDASVNFSE FSRT
(SEQ ID NO: 297)
(297) MGKGDPKKPR GKMSSYAFFV QTCREEHKKK
HPDASVNFSE FSRV
(SEQ ID NO: 298)
(298) MGKGDPKKPR GKMSSYAFFV QTCREEHKKK
HPDASVNFSE FSRW
(SEQ ID NO: 299)
(299) MGKGDPKKPR GKMSSYAFFV QTCREEHKKK
HPDASVNFSE FSRY
(SEQ ID NO: 300)
(300) MGKGDPKKPR GKMSSYAFFV QTCREEHKKK
HPDASVNFSE FSSA
(SEQ ID NO: 301)
(301) MGKGDPKKPR GKMSSYAFFV QTCREEHKKK
HPDASVNFSE FSSC
(SEQ ID NO: 302)
(302) MGKGDPKKPR GKMSSYAFFV QTCREEHKKK
HPDASVNFSE FSSD
(SEQ ID NO: 303)
(303) MGKGDPKKPR GKMSSYAFFV QTCREEHKKK
HPDASVNFSE FSSE
(SEQ ID NO: 304)
(304) MGKGDPKKPR GKMSSYAFFV QTCREEHKKK
HPDASVNFSE FSSF
(SEQ ID NO: 305)
(305) MGKGDPKKPR GKMSSYAFFV QTCREEHKKK
HPDASVNFSE FSSG
(SEQ ID NO: 306)
(306) MGKGDPKKPR GKMSSYAFFV QTCREEHKKK
HPDASVNFSE FSSH
(SEQ ID NO: 307)
(307) MGKGDPKKPR GKMSSYAFFV QTCREEHKKK
HPDASVNFSE FSSI
(SEQ ID NO: 308)
(308) MGKGDPKKPR GKMSSYAFFV QTCREEHKKK
HPDASVNFSE FSSK
(SEQ ID NO: 309)
(309) MGKGDPKKPR GKMSSYAFFV QTCREEHKKK
HPDASVNFSE FSSL
(SEQ ID NO: 310)
(310) MGKGDPKKPR GKMSSYAFFV QTCREEHKKK
HPDASVNFSE FSSM
(SEQ ID NO: 311)
(311) MGKGDPKKPR GKMSSYAFFV QTCREEHKKK
HPDASVNFSE FSSN
(SEQ ID NO: 312)
(312) MGKGDPKKPR GKMSSYAFFV QTCREEHKKK
HPDASVNFSE FSSP
(SEQ ID NO: 313)
(313) MGKGDPKKPR GKMSSYAFFV QTCREEHKKK
HPDASVNFSE FSSQ
(SEQ ID NO: 314)
(314) MGKGDPKKPR GKMSSYAFFV QTCREEHKKK
HPDASVNFSE FSSR
(SEQ ID NO: 315)
(315) MGKGDPKKPR GKMSSYAFFV QTCREEHKKK
HPDASVNFSE FSSS
(SEQ ID NO: 316)
(316) MGKGDPKKPR GKMSSYAFFV QTCREEHKKK
HPDASVNFSE FSST
(SEQ ID NO: 317)
(317) MGKGDPKKPR GKMSSYAFFV QTCREEHKKK
HPDASVNFSE FSSV
(SEQ ID NO: 318)
(318) MGKGDPKKPR GKMSSYAFFV QTCREEHKKK
HPDASVNFSE FSSW
(SEQ ID NO: 319)
(319) MGKGDPKKPR GKMSSYAFFV QTCREEHKKK
HPDASVNFSE FSSY
(SEQ ID NO: 320)
(320) MGKGDPKKPR GKMSSYAFFV QTCREEHKKK
HPDASVNFSE FSTA
(SEQ ID NO: 321)
(321) MGKGDPKKPR GKMSSYAFFV QTCREEHKKK
HPDASVNFSE FSTC
(SEQ ID NO: 322)
(322) MGKGDPKKPR GKMSSYAFFV QTCREEHKKK
HPDASVNFSE FSTD
(SEQ ID NO: 323)
(323) MGKGDPKKPR GKMSSYAFFV QTCREEHKKK
HPDASVNFSE FSTE
(SEQ ID NO: 324)
(324) MGKGDPKKPR GKMSSYAFFV QTCREEHKKK
HPDASVNFSE FSTF
(SEQ ID NO: 325)
(325) MGKGDPKKPR GKMSSYAFFV QTCREEHKKK
HPDASVNFSE FSTG
(SEQ ID NO: 326)
(326) MGKGDPKKPR GKMSSYAFFV QTCREEHKKK
HPDASVNFSE FSTH
(SEQ ID NO: 327)
(327) MGKGDPKKPR GKMSSYAFFV QTCREEHKKK
HPDASVNFSE FSTI
(SEQ ID NO: 328)
(328) MGKGDPKKPR GKMSSYAFFV QTCREEHKKK
HPDASVNFSE FSTK
(SEQ ID NO: 329)
(329) MGKGDPKKPR GKMSSYAFFV QTCREEHKKK
HPDASVNFSE FSTL
(SEQ ID NO: 330)
(330) MGKGDPKKPR GKMSSYAFFV QTCREEHKKK
HPDASVNFSE FSTM
(SEQ ID NO: 331)
(331) MGKGDPKKPR GKMSSYAFFV QTCREEHKKK
HPDASVNFSE FSTN
(SEQ ID NO: 332)
(332) MGKGDPKKPR GKMSSYAFFV QTCREEHKKK
HPDASVNFSE FSTP
(SEQ ID NO: 333)
(333) MGKGDPKKPR GKMSSYAFFV QTCREEHKKK
HPDASVNFSE FSTQ
(SEQ ID NO: 334)
(334) MGKGDPKKPR GKMSSYAFFV QTCREEHKKK
HPDASVNFSE FSTR
(SEQ ID NO: 335)
(335) MGKGDPKKPR GKMSSYAFFV QTCREEHKKK
HPDASVNFSE FSTS
(SEQ ID NO: 336)
(336) MGKGDPKKPR GKMSSYAFFV QTCREEHKKK
HPDASVNFSE FSTT
(SEQ ID NO: 337)
(337) MGKGDPKKPR GKMSSYAFFV QTCREEHKKK
HPDASVNFSE FSTV
(SEQ ID NO: 338)
(338) MGKGDPKKPR GKMSSYAFFV QTCREEHKKK
HPDASVNFSE FSTW
(SEQ ID NO: 339)
(339) MGKGDPKKPR GKMSSYAFFV QTCREEHKKK
HPDASVNFSE FSTY
(SEQ ID NO: 340)
(340) MGKGDPKKPR GKMSSYAFFV QTCREEHKKK
HPDASVNFSE FSVA
(SEQ ID NO: 341)
(341) MGKGDPKKPR GKMSSYAFFV QTCREEHKKK
HPDASVNFSE FSVC
(SEQ ID NO: 342)
(342) MGKGDPKKPR GKMSSYAFFV QTCREEHKKK
HPDASVNFSE FSVD
(SEQ ID NO: 343)
(343) MGKGDPKKPR GKMSSYAFFV QTCREEHKKK
HPDASVNFSE FSVE
(SEQ ID NO: 344)
(344) MGKGDPKKPR GKMSSYAFFV QTCREEHKKK
HPDASVNFSE FSVF
(SEQ ID NO: 345)
(345) MGKGDPKKPR GKMSSYAFFV QTCREEHKKK
HPDASVNFSE FSVG
(SEQ ID NO: 346)
(346) MGKGDPKKPR GKMSSYAFFV QTCREEHKKK
HPDASVNFSE FSVH
(SEQ ID NO: 347)
(347) MGKGDPKKPR GKMSSYAFFV QTCREEHKKK
HPDASVNFSE FSVI
(SEQ ID NO: 348)
(348) MGKGDPKKPR GKMSSYAFFV QTCREEHKKK
HPDASVNFSE FSVK
(SEQ ID NO: 349)
(349) MGKGDPKKPR GKMSSYAFFV QTCREEHKKK
HPDASVNFSE FSVL
(SEQ ID NO: 350)
(350) MGKGDPKKPR GKMSSYAFFV QTCREEHKKK
HPDASVNFSE FSVM
(SEQ ID NO: 351)
(351) MGKGDPKKPR GKMSSYAFFV QTCREEHKKK
HPDASVNFSE FSVN
(SEQ ID NO: 352)
(352) MGKGDPKKPR GKMSSYAFFV QTCREEHKKK
HPDASVNFSE FSVP
(SEQ ID NO: 353)
(353) MGKGDPKKPR GKMSSYAFFV QTCREEHKKK
HPDASVNFSE FSVQ
(SEQ ID NO: 354)
(354) MGKGDPKKPR GKMSSYAFFV QTCREEHKKK
HPDASVNFSE FSVR
(SEQ ID NO: 355)
(355) MGKGDPKKPR GKMSSYAFFV QTCREEHKKK
HPDASVNFSE FSVS
(SEQ ID NO: 356)
(356) MGKGDPKKPR GKMSSYAFFV QTCREEHKKK
HPDASVNFSE FSVT
(SEQ ID NO: 357)
(357) MGKGDPKKPR GKMSSYAFFV QTCREEHKKK
HPDASVNFSE FSVV
(SEQ ID NO: 358)
(358) MGKGDPKKPR GKMSSYAFFV QTCREEHKKK
HPDASVNFSE FSVW
(SEQ ID NO: 359)
(359) MGKGDPKKPR GKMSSYAFFV QTCREEHKKK
HPDASVNFSE FSVY
(SEQ ID NO: 360)
(360) MGKGDPKKPR GKMSSYAFFV QTCREEHKKK
HPDASVNFSE FSWA
(SEQ ID NO: 361)
(361) MGKGDPKKPR GKMSSYAFFV QTCREEHKKK
HPDASVNFSE FSWC
(SEQ ID NO: 362)
(362) MGKGDPKKPR GKMSSYAFFV QTCREEHKKK
HPDASVNFSE FSWD
(SEQ ID NO: 363)
(363) MGKGDPKKPR GKMSSYAFFV QTCREEHKKK
HPDASVNFSE FSWE
(SEQ ID NO: 364)
(364) MGKGDPKKPR GKMSSYAFFV QTCREEHKKK
HPDASVNFSE FSWF
(SEQ ID NO: 365)
(365) MGKGDPKKPR GKMSSYAFFV QTCREEHKKK
HPDASVNFSE FSWG
(SEQ ID NO: 366)
(366) MGKGDPKKPR GKMSSYAFFV QTCREEHKKK
HPDASVNFSE FSWH
(SEQ ID NO: 367)
(367) MGKGDPKKPR GKMSSYAFFV QTCREEHKKK
HPDASVNFSE FSWI
(SEQ ID NO: 368)
(368) MGKGDPKKPR GKMSSYAFFV QTCREEHKKK
HPDASVNFSE FSWK
(SEQ ID NO: 369)
(369) MGKGDPKKPR GKMSSYAFFV QTCREEHKKK
HPDASVNFSE FSWL
(SEQ ID NO: 370)
(370) MGKGDPKKPR GKMSSYAFFV QTCREEHKKK
HPDASVNFSE FSWM
(SEQ ID NO: 371)
(371) MGKGDPKKPR GKMSSYAFFV QTCREEHKKK
HPDASVNFSE FSWN
(SEQ ID NO: 372)
(372) MGKGDPKKPR GKMSSYAFFV QTCREEHKKK
HPDASVNFSE FSWP
(SEQ ID NO: 373)
(373) MGKGDPKKPR GKMSSYAFFV QTCREEHKKK
HPDASVNFSE FSWQ
(SEQ ID NO: 374)
(374) MGKGDPKKPR GKMSSYAFFV QTCREEHKKK
HPDASVNFSE FSWR
(SEQ ID NO: 375)
(375) MGKGDPKKPR GKMSSYAFFV QTCREEHKKK
HPDASVNFSE FSWS
(SEQ ID NO: 376)
(376) MGKGDPKKPR GKMSSYAFFV QTCREEHKKK
HPDASVNFSE FSWT
(SEQ ID NO: 377)
(377) MGKGDPKKPR GKMSSYAFFV QTCREEHKKK
HPDASVNFSE FSWV
(SEQ ID NO: 378)
(378) MGKGDPKKPR GKMSSYAFFV QTCREEHKKK
HPDASVNFSE FSWW
(SEQ ID NO: 379)
(379) MGKGDPKKPR GKMSSYAFFV QTCREEHKKK
HPDASVNFSE FSWY
(SEQ ID NO: 380)
(380) MGKGDPKKPR GKMSSYAFFV QTCREEHKKK
HPDASVNFSE FSYA
(SEQ ID NO: 381)
(381) MGKGDPKKPR GKMSSYAFFV QTCREEHKKK
HPDASVNFSE FSYC
(SEQ ID NO: 382)
(382) MGKGDPKKPR GKMSSYAFFV QTCREEHKKK
HPDASVNFSE FSYD
(SEQ ID NO: 383)
(383) MGKGDPKKPR GKMSSYAFFV QTCREEHKKK
HPDASVNFSE FSYE
(SEQ ID NO: 384)
(384) MGKGDPKKPR GKMSSYAFFV QTCREEHKKK
HPDASVNFSE FSYF
(SEQ ID NO: 385)
(385) MGKGDPKKPR GKMSSYAFFV QTCREEHKKK
HPDASVNFSE FSYG
(SEQ ID NO: 386)
(386) MGKGDPKKPR GKMSSYAFFV QTCREEHKKK
HPDASVNFSE FSYH
(SEQ ID NO: 387)
(387) MGKGDPKKPR GKMSSYAFFV QTCREEHKKK
HPDASVNFSE FSYI
(SEQ ID NO: 388)
(388) MGKGDPKKPR GKMSSYAFFV QTCREEHKKK
HPDASVNFSE FSYK
(SEQ ID NO: 389)
(389) MGKGDPKKPR GKMSSYAFFV QTCREEHKKK
HPDASVNFSE FSYL
(SEQ ID NO: 390)
(390) MGKGDPKKPR GKMSSYAFFV QTCREEHKKK
HPDASVNFSE FSYM
(SEQ ID NO: 391)
(391) MGKGDPKKPR GKMSSYAFFV QTCREEHKKK
HPDASVNFSE FSYN
(SEQ ID NO: 392)
(392) MGKGDPKKPR GKMSSYAFFV QTCREEHKKK
HPDASVNFSE FSYP
(SEQ ID NO: 393)
(393) MGKGDPKKPR GKMSSYAFFV QTCREEHKKK
HPDASVNFSE FSYQ
(SEQ ID NO: 394)
(394) MGKGDPKKPR GKMSSYAFFV QTCREEHKKK
HPDASVNFSE FSYR
(SEQ ID NO: 395)
(395) MGKGDPKKPR GKMSSYAFFV QTCREEHKKK
HPDASVNFSE FSYS
(SEQ ID NO: 396)
(396) MGKGDPKKPR GKMSSYAFFV QTCREEHKKK
HPDASVNFSE FSYT
(SEQ ID NO: 397)
(397) MGKGDPKKPR GKMSSYAFFV QTCREEHKKK
HPDASVNFSE FSYV
(SEQ ID NO: 398)
(398) MGKGDPKKPR GKMSSYAFFV QTCREEHKKK
HPDASVNFSE FSYW
(SEQ ID NO: 399)
(399) MGKGDPKKPR GKMSSYAFFV QTCREEHKKK
HPDASVNFSE FSYY
(SEQ ID NO: 403)
(400) GKGDPKKPR GKMSSYAFFV QTCREEHKKK HPDASVNFSE
FSPP

The peptide of the present invention further includes a peptide having substitution of an amino acid at position 43 or/and position 44 of the peptide shown in the above SEQ ID NOs: 1 to 399 with a non-naturally occurring amino acid, and a peptide having substitution of an amino acid at position 42 or/and position 43 of the peptide shown in the above SEQ ID NO: 403 with a non-naturally occurring amino acid.

The peptide of the present invention may have a conservative substitution of an amino acid or amino acids in the peptide having the amino acid sequence shown in any one of SEQ ID NOs: 1 to 5. The conservative substitution refers to substitution with another amino acid having a structure or properties chemically similar to those of the original amino acid, without undergoing substantial changes in the functions of the peptide. Examples of the conservative substitution include mutual substitutions between aspartic acid and glutamic acid which are acidic amino acids; mutual substitutions between lysine, arginine, and histidine which are basic amino acids; mutual substitutions between serine, threonine, asparagine, and glutamine which are hydrophilic amino acids; and the like. For example, a peptide of SEQ ID NO: 167 is a product in which glutamic acid (E) at position 44 of the peptide of SEQ ID NO: 5 is substituted with aspartic acid (D), which is considered to have a function equivalent to that of the peptide of SEQ ID NO: 5. Further, peptides shown in SEQ ID NOs: 126, 128, 129, 138, 248, 254, 280, 282, 283 and 292 also have a conservative substitution of the amino acid in the peptide having the amino acid sequence shown in any one of SEQ ID NOs: 1 to 5, which are considered to have the function equivalent to that of any one of the peptide of SEQ ID NOs: 1 to 5. In one embodiment, the peptide of the present invention has the amino acid sequence shown in any one of SEQ ID NOs: 1 to 5, 126, 128, 129, 138, 167, 248, 254, 280, 282, 283, 292 and 403. In one more preferred embodiment, the peptide of the present invention has the amino acid sequence shown in any one of SEQ ID NOs: 1 to 5, 167 and 403, and in one even more preferred embodiment, the peptide of the present invention has the amino acid sequence shown in any one of SEQ ID NOs: 1 to 5 and 403, from the viewpoint that the peptide is expected to have the above function more surely.

Therefore, one of preferred embodiments of the peptide defined in the above (a) is a peptide having the amino acid sequence shown in any one of SEQ ID NOs: 1 to 5, 126, 128, 129, 138, 167, 248, 254, 280, 282, 283, 292 and 403, one of more preferred embodiment is a peptide having the amino acid sequence shown in any one of SEQ ID NOs: 1 to 5, 167 and 403, and one of even more preferred embodiment is a peptide having the amino acid sequence shown in any one of SEQ ID NOs: 1 to 5 and 403.

Further, one of preferred embodiments of the peptide defined in the above (b) is a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5, 126, 128, 129, 138, 167, 248, 254, 280, 282, 283, 292 and 403, and having an activity of stimulating migration of cells, one of more preferred embodiment is a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5, 167 and 403, and having an activity of stimulating migration of cells, and one of even more preferred embodiment is a peptide consisting of an amino acid sequence having addition of one or more amino acids, e.g., 2 to 40 amino acids, 2 to 30 amino acids, 2 to 20 amino acids, 2 to 10 amino acids, 2 to 5 amino acids, or 4, 3, or 2 amino acids, to the peptide having the amino acid sequence shown in any one of SEQ ID NOs: 1 to 5 and 403, and having an activity of stimulating migration of cells.

Further, one of preferred embodiments of the peptide as defined in the above (c) is a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5, 126, 128, 129, 138, 167, 248, 254, 280, 282, 283 and 292 or an amino acid of position 1 to position 41 of the peptide having the amino acid sequence shown in SEQ ID NO: 403, and having an activity of stimulating migration of cells, one of more preferred embodiments is a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5 and 167 or an amino acid of position 1 to position 41 of the peptide having the amino acid sequence shown in SEQ ID NO: 403, and having an activity of stimulating migration of cells, and one of even more preferred embodiments is a peptide comprising or consisting of an amino acid sequence having substitution, deletion, or insertion of one or more amino acids, e.g., 2 to 40 amino acids, 2 to 30 amino acids, 2 to 20 amino acids, 2 to 10 amino acids, 2 to 5 amino acids, or 4, 3, or 2 amino acids, at an amino acid of position 1 to position 42 of the peptide having the amino acid sequence shown in any one of SEQ ID NOs: 1 to 5 or an amino acid of position 1 to position 41 of the peptide having the amino acid sequence shown in SEQ ID NO: 403, and having an activity of stimulating migration of cells.

The term “derivative” of the peptide of the present invention means a peptide having substantially the same biological function or activity as the peptide of the present invention, in other words, the activity of stimulating migration of cells. The C-terminal of the derivative of the peptide may be any one of a carboxyl group, a carboxylate, an amide, or an ester. For example, the derivative includes a modified peptide obtained by addition of a modifying group, a mutant peptide obtained by altering an amino acid residue, and the like.

The modifying group includes functional groups showing fluorescent properties, functional groups not involved in the formation of conformation of a peptide, and the like. In addition, the modifying group may be an amino acid residue (for example, β-alanine etc.) other than an amino acid residue constituting the naturally occurring peptide of the present invention. The functional group showing fluorescent properties includes eosin, fluorescein isothiocyanate (FITC) and the like. The functional groups not involved in the formation of conformation of a peptide include spacer groups represented by β-alanine residues, and the like. It is preferable that the functional group is present at a terminal. Further, a peptide modified with polyethylene glycol (PEG) is also included in the “derivative.”

The term “pharmaceutically acceptable salt” of the peptide of the present invention includes physiologically acceptable salts with acids or bases, and particularly acid-added salts are preferred. As the salt as mentioned above, a salt with an inorganic acid such as hydrochloric acid, phosphoric acid, hydrobromic acid, or sulfuric acid, a salt with an organic acid such as acetic acid, formic acid, propionic acid, fumaric acid, maleic acid, succinic acid, tartaric acid, citric acid, malic acid, oxalic acid, benzoic acid, methanesulfonic acid, or benzenesulfonic acid, or the like is used.

The term “solvate” of the peptide of the present invention or the above salt includes those in which any number of solvent molecules are coordinated to the peptide of the present invention, and preferably a hydrate.

The present invention provides the peptide as described above. In the synthesis of the peptide in the present invention, the peptide can be chemically synthesized according to any of the methods of the peptide liquid phase synthesis methods and the peptide solid phase synthesis methods. In the present invention, the peptide synthesized according to the peptide solid phase synthesis method is preferred. The peptide solid phase synthesis method is one of the generally employed methods when peptides are chemically synthesized, which can be, for example, carried out as follows: Using as a solid phase beads of polystyrene polymer gel having a diameter of 0.1 mm or so of which surfaces are modified with amino groups, an amino acid chain is extended one by one by dehydration reaction therefrom. When an intended sequence of the peptide is prepared, the peptide is cut out from the solid phase surface, to obtain an intended substance.

According to the solid phase synthesis method, it is made possible to perform the synthesis of ribosomal peptide which is difficult to synthesize in bacteria, introduction of a non-naturally occurring amino acid such as D-form or heavy atom substituted compound, modification of peptide and protein main chain, or the like. When a long peptide chain of from 70 to exceeding 100 peptides is synthesized in the solid phase method, the peptide chains can be synthesized by binding two peptide chains using a native chemical ligation method.

The method for administration of a composition of the present invention includes oral administration or parenteral administration, and specific methods of the parenteral administration include injection administration, transnasal administration, transpulmonary administration, transdermal administration, and the like, but not limited thereto. As examples of the injection administration, for example, a composition of the present invention can be administered systemically or locally (e.g., subcutaneously, intradermally, epidermally, to eyeball or palpebral conjunctiva, to nasal cavity mucosa, to oral cavity and gastrointestinal mucosa, to vaginal or intrauterine mucosa, to a damaged site, etc.) by intravenous injection, intramuscular injection, intraperitoneal injection, subcutaneous injection or the like.

In addition, the method for administration of a composition of the present invention includes, for example, intravascular administration (intraarterial administration, intravenous administration, etc.), blood administration, intramuscular administration, subcutaneous administration, intradermal administration, and intraperitoneal administration, but not limited thereto.

In addition, the site of administration is not limited, which includes a tissue site in need of regeneration or its neighborhood region, a site different from the tissue in need of regeneration, or a site distal and different from the tissue in need of regeneration. For example, the site of administration includes blood (intraarterially, intravenously, etc.), muscles, subcutaneous site, intradermal site and intraperitoneal site, but not limited thereto.

In addition, the method for administration can be appropriately selected depending upon age and symptoms of the patients. When the peptide of the present invention is administered, for example, a dose within a range of from 0.0000001 mg to 1,000 mg per 1 kg body weight per single administration can be selected. Alternatively, for example, a dose within a range of from 0.00001 to 100,000 mg/body per patient can be selected. When cells that secrete the peptide of the present invention or a vector for genetic therapy into which a DNA encoding the peptide is inserted is administered, the peptide can be administered such that the amount of the peptide is within the above range. However, the pharmaceutical composition of the present invention is not limited to these doses.

The pharmaceutical composition of the present invention can be formulated in accordance with conventional methods (e.g., Remington's Pharmaceutical Science, latest edition, Mark Publishing Company, Easton, U.S.A), which may together contain a pharmaceutically acceptable carrier or additives. Examples of the carriers and additives include, for example, surfactants, excipients, colorants, perfumes, preservatives, stabilizers, buffers, suspending agents, isotonic agents, binders, disintegrants, lubricants, fluidity promoters, flavoring agents, and the like, but not limited thereto, and other conventionally used carriers can be appropriately used. Specific examples include light anhydrous silicic acid, lactose, crystalline celluloses, mannitol, starches, carmellose calcium, carmellose sodium, hydroxypropyl cellulose, hydroxypropylmethyl cellulose, polyvinyl acetal diethylaminoacetate, polyvinyl pyrrolidone, gelatin, medium-chained fatty acid triglyceride, polyoxyethylene hydrogenated castor oil 60, white sugar, carboxymethyl cellulose, corn starch, inorganic salts, and the like.

Here, all the prior art references cited in the present specification will be incorporated herein by reference.

### EXAMPLES

The present invention will be further illustrated by means of the following Examples, without intending to limit the present invention to the following Examples.

### Example 1

### Synthesis of Various Kinds of HMGB1 Peptides

A peptide consisting of the amino acids at position 2 to position 44 of human HMGB1 (2-44) and each of the five kinds of peptides having substitution of an amino acid sequence at position 43 and/or position 44 in the peptide at position 1 to position 44 of human HMGB1 (Peptide-1 to -5) were synthesized by a solid phase method.

2-44
(SEQ ID NO: 400)
GKGDPKKPR GKMSSYAFFV QTCREEHKKK
HPDASVNFSE FSKK
Peptide-1
(SEQ ID NO: 1)
MGKGDPKKPR GKMSSYAFFV QTCREEHKKK
HPDASVNFSE FSKP
Peptide-2
(SEQ ID NO: 2)
MGKGDPKKPR GKMSSYAFFV QTCREEHKKK
HPDASVNFSE FSPK
Peptide-3
(SEQ ID NO: 3)
MGKGDPKKPR GKMSSYAFFV QTCREEHKKK
HPDASVNFSE FSPP
Peptide-4
(SEQ ID NO: 4)
MGKGDPKKPR GKMSSYAFFV QTCREEHKKK
HPDASVNFSE FSKA
Peptide-5
(SEQ ID NO: 5)
MGKGDPKKPR GKMSSYAFFV QTCREEHKKK
HPDASVNFSE FSKE

### Example 2

### Measurements of Cell-Migration Activity

Cells of murine bone marrow-derived stromal cell line ST2 (RCB0224, RIKEN BioResource Research Center) were removed from a dish using TrypLE Express (Thermo Fisher Scientific), and collected by centrifugation at 25° C. and 1,500 rpm for 5 minutes. The cell pellets were prepared into a concentration of 7×10⁵ cells/mL with RPMI 1640/10% FBS/1% PenStrep (Gibco), and seeded at 70 μL/insert well on a 24-well plate set with a culture insert (Nippon Genetics Co., Ltd.). After 24 hours, the culture insert was taken out, and the cells were washed once with RPMI 1640. Each of the peptide samples (a peptide of position 2 to position 44 of human HMGB1 (2-44), and the five kinds of modified peptides Peptide-1 to -5 prepared in Example 1), each prepared at 10 μM with an assay medium (RPMI 1640/0.1% FBS) was added in an amount of 550 μL, and the cells were further cultured overnight. As a negative control (NC), only the assay medium was added. After the culture, the cells were fixed, stained with DiffQuik, and photographed. The degree of cell migration of each group from the photographed images was evaluated with five levels of scores (−, ±, +, ++, +++). In addition, each of the peptide samples was subjected to three test runs under the same conditions.

Results

The presence or absence of the migration activity of the peptide of position 2 to position 44 of human HMGB1 (2-44), the five kinds of modified peptides Peptide-1 to -5 prepared in Example 1, and NC was ascertained. As a result, all of the five kinds of modified peptides were found to have migration activity (Table 1).

TABLE 1

| | 1st Run | 2nd Run | 3rd Run |
|---|---|---|---|
| NC | − | ± | − |
| 2-44 | +++ | +++ | +++ |
| Peptide-1 | +++ | + | +++ |
| Peptide-2 | +++ | ++ | +++ |
| Peptide-3 | + | + | + |
| Peptide-4 | ++ | + | +++ |
| Peptide-5 | ++ | ++ | ++ |

### Example 3

### Stability of Peptides in Plasma

Whole blood collected from Crl: CD (SD) rats using an anti-coagulant EDTA-2K was centrifuged to obtain a blank plasma. The plasma obtained was stored on ice, and used only on the day of the stability test.

Each of the six kinds of peptides used in Example 2 was prepared into a solution at 375 μg/mL with distilled water from a stock solution in which each peptide was dissolved in a 0.1% aqueous TFA solution at 1 mg/mL. Sixteen microliters of the solution was added to 1,184 μL of the blank plasma to prepare a sample for the stability test (assay concentration: 5 μg/mL). The sample for stability test was allowed to stand on ice, and 50 μL (at each time point, 3 samples) was collected immediately after the addition, and 1, 3, 5, 15, 30, and 60 minutes after the addition. To 50 μL of the collected sample were added 250 μL of 270 mmol/L citric acid solution/130 mmol/L EDTA-2K aqueous solution (4:1, v/v), 25 μL of a 0.1% aqueous TFA solution, and 25 μL of IS solution (4 μg/mL aqueous 0.1% TFA solution of a compound in which nine carbon atoms and one nitrogen atom of phenylalanine at position 18, position 38, and position 41 in the peptide of position 1 to position 44 of human HMGB1 are each substituted with a stable isotope), and mixed. To the mixture was added 350 μL of methanol and mixed, and further 100 μL of acetonitrile was added thereto and mixed, and the mixture was once frozen with dry ice. The frozen mixture was thawed at room temperature, and the supernatant obtained by centrifugation was transferred to a separate container and stirred to be used as a sample for the measurement.

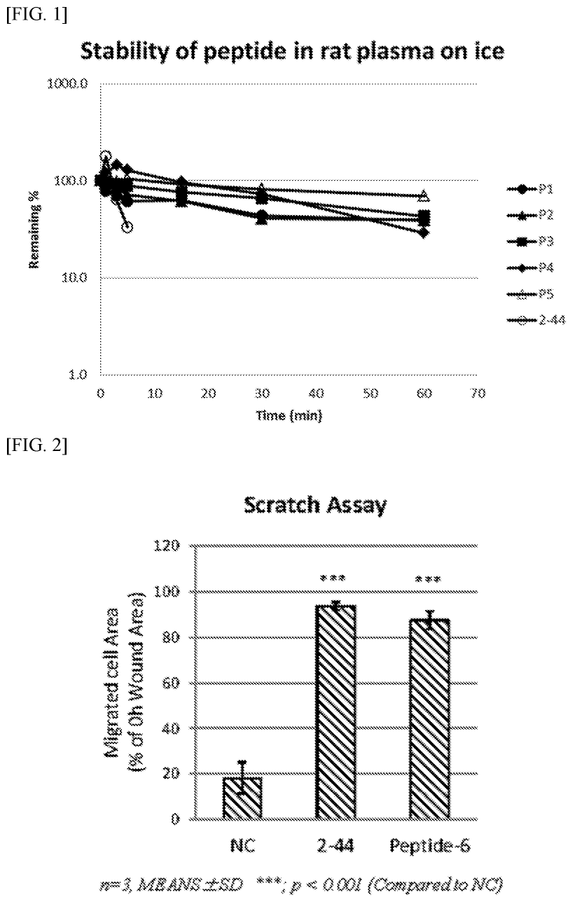

The samples for the measurement were introduced into high-performance liquid chromatograph-tandem quadrupolar mass spectrometer to detect the monitor ions peculiar to each of the peptides. From separately prepared samples of which concentrations were known, a calibration curve was drawn in a concentration range of from 0.1 to 10 μg/mL, and an inverse regression was obtained from peak areas of chromatograms obtained by measurements of each sample to obtain a peptide concentration in the samples. The peptide concentration at each time point was averaged, and a remaining rate (%) which was calculated by defining one immediately after the addition as 100 was plotted against the time after the addition (FIG. 1).

### Results

As a result of the stability test in rat plasma (on ice) of the peptide at position 2 to position 44 of human HMGB1 (2-44) and the five kinds of peptides in which the amino acids at position 43 and position 44 were modified (Peptide-1 to -5), 2-44 were equal to or lower than the quantifiable lower limit (0.5 μg/mL) for the sample at 15 minutes or more later of the addition. On the other hand, it was confirmed that the five kinds of the modified peptides Peptide-1 to -5 all were stable as compared to 2-44 (FIG. 1).

### Example 4

### Synthesis of HMGB1 Peptide

A peptide consisting of a sequence given below having substitution of two amino acids at a C-terminal in the peptide of position 2 to position 44 of human HMGB1 (2-44) (Peptide-6) was synthesized by a solid phase method.

Peptide-6 GKGDPKKPR GKMSSYAFFV QTCREEHKKK HPDASVNFSE FSPP (SEQ ID NO: 403)

### Example 5

### Measurements of Cell-Migration Activity

Cells of murine bone marrow-derived stromal cell line ST2 (RCB0224, RIKEN BioResource Research Center) were removed from a dish using 2.5 g/L—Trypsin/lmmol/L—EDTA Solution (nacalai tesque), and collected by centrifugation at 25° C. and 200 g for 3 minutes. The cell pellets were prepared into a concentration of 8.6×10⁵ cells/mL with RPMI 1640/10% FBS/1% PenStrep (nacalai tesque), and seeded at 70 μL/insert well on a 24-well plate set with a culture insert (Nippon Genetics Co., Ltd.). After 18 hours, the culture insert was taken out, and the cells were washed once with RPMI 1640. Each of the peptide samples (the peptide of position 2 to position 44 of human HMGB1 (2-44), and the modified peptide Peptide-6 prepared in Example 4), each prepared at 20 μM with an assay medium (RPMI 1640/0.1% FBS) was added in an amount of 1,0000μL, and the cells were cultured for additional 20 hours. As a negative control (NC), only the assay medium was added. Immediately after the addition of the assay medium and after 20 hours of culture, the cells were photographed. From the photographed image, the degree of cell migration of each group was evaluated as a proportion (%) of the area occupied by the cells migrated to the region at 20 hours relative to the area of blank region prepared by the culture insert (region in which the cells do not exist at the time point of addition of the assay medium).

### Results

The presence or absence of the migration activity of the peptide at position 2 to position 44 of human HMGB1 (2-44), the modified peptide prepared in Example 4 (Peptide-6) and NC was ascertained. As a result, the modified peptide (Peptide-6) was found to have the same level of migration activity as the peptide at position 2 to position 44 of human HMGB1 (2-44) (FIG. 2).

### Example 6

### Stability of Peptides in Plasma

A 5 mL tube previously charged with 60 μL of a 10% EDTA-2K was charged with 3 mL of whole blood collected from Crl: CD (SD) rats, and the contents were allowed to stand at room temperature for 30 minutes, and then centrifuged at 4° C. and at 3,000 rpm (1600×g) for 10 minutes. The supernatant was collected to be used as a blank plasma. The plasma obtained was stored on ice and only used on the day of the stability test.

Each of the peptide at position 2 to position 44 of human HMGB1 (2-44), the modified peptide prepared in Example 1 (Peptide-3), and the modified peptide prepared in Example 4 (Peptide-6) was prepared into a solution at 375 μg/mL with distilled water from a stock solution in which each peptide was dissolved in a 0.1% aqueous TFA solution at 1 mg/mL. Eight microliters of the solution was added to 592 μL of the blank plasma to prepare a sample for the stability test (assay concentration: 5 μg/mL). The sample for stability test was allowed to stand on ice, and 50 μL was collected immediately after the addition, and 30 and 60 minutes after the addition. To 50 μL of the collected sample were added 250 μL of 270 mmol/L citric acid solution/130 mmol/L EDTA-2K aqueous solution (4:1, v/v), and 50 μL of a 0.1% aqueous TFA solution, and mixed. To the mixture was added 350 μL of methanol and mixed, and further 100 μL of acetonitrile was added thereto and mixed, and the mixture was once frozen with dry ice. After 5 to 60 minutes, the frozen sample was thawed at room temperature, and the supernatant obtained by centrifugation at 4° C. and at 14,000×g for 3 minutes was collected in an amount of 400 μL, dried and solidified with a centrifugal concentrator (Spin Dryer Light VC-36R, manufactured by TAITEC CORPORATION), and dissolved in 200 μL of a 1% TFA/2% ACN Buffer, and filtrated with a filter (0.22 μm) to obtain samples for the measurement.

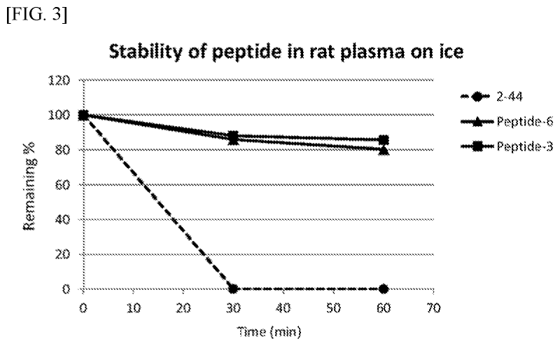

The samples for the measurement were introduced into high-performance liquid chromatograph-single quadrupolar mass spectrometer to detect the monitor ions peculiar to each of the peptides. From separately prepared samples of which concentrations were known, a calibration curve was drawn in a concentration range of from 0.1 to 10 μg/mL, and an inverse regression was obtained from peak areas of chromatograms obtained by measurements of each sample to obtain a peptide concentration in the samples. For the peptide concentration at each time point, a remaining rate (%) which was calculated by defining one immediately after the addition as 100 was plotted against the time after the addition (FIG. 3).

### Results

As a result of the stability test in rat plasma (on ice), 2-44 showed to be equal to or lower than the quantifiable lower limit (0.5 μg/mL) at a time point of 30 minutes after the addition. On the other hand, it was confirmed that both of Peptide-3 and Peptide-6 were stable as compared to 2-44. In addition, the stability of Peptide-3 and Peptide-6 was found to be of the same level (FIG. 3).

### INDUSTRIAL APPLICABILITY

The peptide, a derivative thereof, a pharmaceutically acceptable salt thereof, or a solvate thereof of the present invention is used in the field of regenerative medicine.

1. A peptide selected from the group consisting of:
a peptide having an amino acid sequence shown in any one of SEQ ID NOs: 1 to 399 and 403,
a peptide consisting of an amino acid sequence having an addition of one or more amino acids to the peptide having the amino acid sequence shown in any one of SEQ ID NOs: 1 to 399 and 403, and having an activity of stimulating migration of cells, and
a peptide comprising or consisting of an amino acid sequence having a substitution, deletion, or insertion of one or more amino acids at an amino acid of position 1 to position 42 of the peptide having the amino acid sequence shown in any one of SEQ ID NOs: 1 to 399 or at an amino acid of position 1 to position 41 of the peptide having the amino acid sequence shown in SEQ ID NO: 403, and having an activity of stimulating migration of cells,
a derivative thereof, a pharmaceutically acceptable salt thereof, or a solvate thereof.

2. A peptide selected from the group consisting of:
a peptide having an amino acid sequence shown in any one of SEQ ID NOs: 1 to 5, 126, 128, 129, 138, 167, 248, 254, 280, 282, 283, 292 and 403,
a peptide consisting of an amino acid sequence having an addition of one or more amino acids to the peptide having the amino acid sequence shown in any one of SEQ ID NOs: 1 to 5, 126, 128, 129, 138, 167, 248, 254, 280, 282, 283, 292 and 403, and having an activity of stimulating migration of cells, and
a peptide comprising or consisting of an amino acid sequence having a substitution, deletion, or insertion of one or more amino acids at an amino acid of position 1 to position 42 of the peptide having the amino acid sequence shown in any one of SEQ ID NOs: 1 to 5, 126, 128, 129, 138, 167, 248, 254, 280, 282, 283 and 292, or at an amino acid of position 1 to position 41 of the peptide having the amino acid sequence shown in SEQ ID NO: 403, and having an activity of stimulating migration of cells,
a derivative thereof, a pharmaceutically acceptable salt thereof, or a solvate thereof.

3. A peptide selected from the group consisting of:
a peptide having an amino acid sequence shown in any one of SEQ ID NOs: 1 to 5, 167 and 403,
a peptide consisting of an amino acid sequence having an addition of one or more amino acids to the peptide having the amino acid sequence shown in any one of SEQ ID NOs: 1 to 5, 167 and 403, and having an activity of stimulating migration of cells, and
a peptide comprising or consisting of an amino acid sequence having a substitution, deletion, or insertion of one or more amino acids at an amino acid of position 1 to position 42 of the peptide having the amino acid sequence shown in any one of SEQ ID NOs: 1 to 5 and 167 or at an amino acid of position 1 to position 41 of the peptide having the amino acid sequence shown in SEQ ID NO: 403, and having an activity of stimulating migration of cells,
a derivative thereof, a pharmaceutically acceptable salt thereof, or a solvate thereof.

4. A peptide selected from the group consisting of:
a peptide having an amino acid sequence shown in any one of SEQ ID NOs: 1 to 5 and 403,
a peptide consisting of an amino acid sequence having an addition of one or more amino acids to the peptide having the amino acid sequence shown in any one of SEQ ID NOs: 1 to 5 and 403, and having an activity of stimulating migration of cells, and
a peptide comprising or consisting of an amino acid sequence having g substitution, deletion, or insertion of one or more amino acids at an amino acid of position 1 to position 42 of the peptide having the amino acid sequence shown in any one of SEQ ID NOs: 1 to 5 or at an amino acid of position 1 to position 41 of the peptide having the amino acid sequence shown in SEQ ID NO: 403, and having an activity of stimulating migration of cells,
a derivative thereof, a pharmaceutically acceptable salt thereof, or a solvate thereof.

5. A method for stimulating migration of bone marrow-derived stromal cells in a subject in need thereof, comprising administering the peptide, derivative thereof, pharmaceutically acceptable salt thereof, or solvate thereof according to claim 1 to the subject.

6. A method for stimulating migration of bone marrow-derived stromal cells in a subject in need thereof, comprising administering the peptide, derivative thereof, pharmaceutically acceptable salt thereof, or solvate thereof according to claim 2 to the subject.

7. A method for stimulating migration of bone marrow-derived stromal cells in a subject in need thereof, comprising administering the peptide, derivative thereof, pharmaceutically acceptable salt thereof, or solvate thereof according to claim 3 to the subject.

8. A method for stimulating migration of bone marrow-derived stromal cells in a subject in need thereof, comprising administering the peptide, derivative thereof, pharmaceutically acceptable salt thereof, or solvate thereof according, to claim 4 to the subject.


SEQUENCE LISTING (403 sequences)

SEQ ID NO: 1 - protein, 44 aa, Artificial Sequence
  Synthetic
MGKGDPKKPRGKMSSYAFFVQTCREEHKKKHPDASVNFSEFSKP

SEQ ID NO: 2 - protein, 44 aa, Artificial Sequence
  Synthetic
MGKGDPKKPRGKMSSYAFFVQTCREEHKKKHPDASVNFSEFSPK

SEQ ID NO: 3 - protein, 44 aa, Artificial Sequence
  Synthetic
MGKGDPKKPRGKMSSYAFFVQTCREEHKKKHPDASVNFSEFSPP

SEQ ID NO: 4 - protein, 44 aa, Artificial Sequence
  Synthetic
MGKGDPKKPRGKMSSYAFFVQTCREEHKKKHPDASVNFSEFSKA

SEQ ID NO: 5 - protein, 44 aa, Artificial Sequence
  Synthetic
MGKGDPKKPRGKMSSYAFFVQTCREEHKKKHPDASVNFSEFSKE

SEQ ID NO: 6 - protein, 44 aa, Artificial Sequence
  Synthetic
MGKGDPKKPRGKMSSYAFFVQTCREEHKKKHPDASVNFSEFSAA

SEQ ID NO: 7 - protein, 44 aa, Artificial Sequence
  Synthetic
MGKGDPKKPRGKMSSYAFFVQTCREEHKKKHPDASVNFSEFSAC

SEQ ID NO: 8 - protein, 44 aa, Artificial Sequence
  Synthetic
MGKGDPKKPRGKMSSYAFFVQTCREEHKKKHPDASVNFSEFSAD

SEQ ID NO: 9 - protein, 44 aa, Artificial Sequence
  Synthetic
MGKGDPKKPRGKMSSYAFFVQTCREEHKKKHPDASVNFSEFSAE

SEQ ID NO: 10 - protein, 44 aa, Artificial Sequence
  Synthetic
MGKGDPKKPRGKMSSYAFFVQTCREEHKKKHPDASVNFSEFSAF

SEQ ID NO: 11 - protein, 44 aa, Artificial Sequence
  Synthetic
MGKGDPKKPRGKMSSYAFFVQTCREEHKKKHPDASVNFSEFSAG

SEQ ID NO: 12 - protein, 44 aa, Artificial Sequence
  Synthetic
MGKGDPKKPRGKMSSYAFFVQTCREEHKKKHPDASVNFSEFSAH

SEQ ID NO: 13 - protein, 44 aa, Artificial Sequence
  Synthetic
MGKGDPKKPRGKMSSYAFFVQTCREEHKKKHPDASVNFSEFSAI

SEQ ID NO: 14 - protein, 44 aa, Artificial Sequence
  Synthetic
MGKGDPKKPRGKMSSYAFFVQTCREEHKKKHPDASVNFSEFSAK

SEQ ID NO: 15 - protein, 44 aa, Artificial Sequence
  Synthetic
MGKGDPKKPRGKMSSYAFFVQTCREEHKKKHPDASVNFSEFSAL

SEQ ID NO: 16 - protein, 44 aa, Artificial Sequence
  Synthetic
MGKGDPKKPRGKMSSYAFFVQTCREEHKKKHPDASVNFSEFSAM

SEQ ID NO: 17 - protein, 44 aa, Artificial Sequence
  Synthetic
MGKGDPKKPRGKMSSYAFFVQTCREEHKKKHPDASVNFSEFSAN

SEQ ID NO: 18 - protein, 44 aa, Artificial Sequence
  Synthetic
MGKGDPKKPRGKMSSYAFFVQTCREEHKKKHPDASVNFSEFSAP

SEQ ID NO: 19 - protein, 44 aa, Artificial Sequence
  Synthetic
MGKGDPKKPRGKMSSYAFFVQTCREEHKKKHPDASVNFSEFSAQ

SEQ ID NO: 20 - protein, 44 aa, Artificial Sequence
  Synthetic
MGKGDPKKPRGKMSSYAFFVQTCREEHKKKHPDASVNFSEFSAR

SEQ ID NO: 21 - protein, 44 aa, Artificial Sequence
  Synthetic
MGKGDPKKPRGKMSSYAFFVQTCREEHKKKHPDASVNFSEFSAS

SEQ ID NO: 22 - protein, 44 aa, Artificial Sequence
  Synthetic
MGKGDPKKPRGKMSSYAFFVQTCREEHKKKHPDASVNFSEFSAT

SEQ ID NO: 23 - protein, 44 aa, Artificial Sequence
  Synthetic
MGKGDPKKPRGKMSSYAFFVQTCREEHKKKHPDASVNFSEFSAV

SEQ ID NO: 24 - protein, 44 aa, Artificial Sequence
  Synthetic
MGKGDPKKPRGKMSSYAFFVQTCREEHKKKHPDASVNFSEFSAW

SEQ ID NO: 25 - protein, 44 aa, Artificial Sequence
  Synthetic
MGKGDPKKPRGKMSSYAFFVQTCREEHKKKHPDASVNFSEFSAY

SEQ ID NO: 26 - protein, 44 aa, Artificial Sequence
  Synthetic
MGKGDPKKPRGKMSSYAFFVQTCREEHKKKHPDASVNFSEFSCA

SEQ ID NO: 27 - protein, 44 aa, Artificial Sequence
  Synthetic
MGKGDPKKPRGKMSSYAFFVQTCREEHKKKHPDASVNFSEFSCC

SEQ ID NO: 28 - protein, 44 aa, Artificial Sequence
  Synthetic
MGKGDPKKPRGKMSSYAFFVQTCREEHKKKHPDASVNFSEFSCD

SEQ ID NO: 29 - protein, 44 aa, Artificial Sequence
  Synthetic
MGKGDPKKPRGKMSSYAFFVQTCREEHKKKHPDASVNFSEFSCE

SEQ ID NO: 30 - protein, 44 aa, Artificial Sequence
  Synthetic
MGKGDPKKPRGKMSSYAFFVQTCREEHKKKHPDASVNFSEFSCF

SEQ ID NO: 31 - protein, 44 aa, Artificial Sequence
  Synthetic
MGKGDPKKPRGKMSSYAFFVQTCREEHKKKHPDASVNFSEFSCG

SEQ ID NO: 32 - protein, 44 aa, Artificial Sequence
  Synthetic
MGKGDPKKPRGKMSSYAFFVQTCREEHKKKHPDASVNFSEFSCH

SEQ ID NO: 33 - protein, 44 aa, Artificial Sequence
  Synthetic
MGKGDPKKPRGKMSSYAFFVQTCREEHKKKHPDASVNFSEFSCI

SEQ ID NO: 34 - protein, 44 aa, Artificial Sequence
  Synthetic
MGKGDPKKPRGKMSSYAFFVQTCREEHKKKHPDASVNFSEFSCK

SEQ ID NO: 35 - protein, 44 aa, Artificial Sequence
  Synthetic
MGKGDPKKPRGKMSSYAFFVQTCREEHKKKHPDASVNFSEFSCL

SEQ ID NO: 36 - protein, 44 aa, Artificial Sequence
  Synthetic
MGKGDPKKPRGKMSSYAFFVQTCREEHKKKHPDASVNFSEFSCM

SEQ ID NO: 37 - protein, 44 aa, Artificial Sequence
  Synthetic
MGKGDPKKPRGKMSSYAFFVQTCREEHKKKHPDASVNFSEFSCN

SEQ ID NO: 38 - protein, 44 aa, Artificial Sequence
  Synthetic
MGKGDPKKPRGKMSSYAFFVQTCREEHKKKHPDASVNFSEFSCP

SEQ ID NO: 39 - protein, 44 aa, Artificial Sequence
  Synthetic
MGKGDPKKPRGKMSSYAFFVQTCREEHKKKHPDASVNFSEFSCQ

SEQ ID NO: 40 - protein, 44 aa, Artificial Sequence
  Synthetic
MGKGDPKKPRGKMSSYAFFVQTCREEHKKKHPDASVNFSEFSCR

SEQ ID NO: 41 - protein, 44 aa, Artificial Sequence
  Synthetic
MGKGDPKKPRGKMSSYAFFVQTCREEHKKKHPDASVNFSEFSCS

SEQ ID NO: 42 - protein, 44 aa, Artificial Sequence
  Synthetic
MGKGDPKKPRGKMSSYAFFVQTCREEHKKKHPDASVNFSEFSCT

SEQ ID NO: 43 - protein, 44 aa, Artificial Sequence
  Synthetic
MGKGDPKKPRGKMSSYAFFVQTCREEHKKKHPDASVNFSEFSCV

SEQ ID NO: 44 - protein, 44 aa, Artificial Sequence
  Synthetic
MGKGDPKKPRGKMSSYAFFVQTCREEHKKKHPDASVNFSEFSCW

SEQ ID NO: 45 - protein, 44 aa, Artificial Sequence
  Synthetic
MGKGDPKKPRGKMSSYAFFVQTCREEHKKKHPDASVNFSEFSCY

SEQ ID NO: 46 - protein, 44 aa, Artificial Sequence
  Synthetic
MGKGDPKKPRGKMSSYAFFVQTCREEHKKKHPDASVNFSEFSDA

SEQ ID NO: 47 - protein, 44 aa, Artificial Sequence
  Synthetic
MGKGDPKKPRGKMSSYAFFVQTCREEHKKKHPDASVNFSEFSDC

SEQ ID NO: 48 - protein, 44 aa, Artificial Sequence
  Synthetic
MGKGDPKKPRGKMSSYAFFVQTCREEHKKKHPDASVNFSEFSDD

SEQ ID NO: 49 - protein, 44 aa, Artificial Sequence
  Synthetic
MGKGDPKKPRGKMSSYAFFVQTCREEHKKKHPDASVNFSEFSDE

SEQ ID NO: 50 - protein, 44 aa, Artificial Sequence
  Synthetic
MGKGDPKKPRGKMSSYAFFVQTCREEHKKKHPDASVNFSEFSDF

SEQ ID NO: 51 - protein, 44 aa, Artificial Sequence
  Synthetic
MGKGDPKKPRGKMSSYAFFVQTCREEHKKKHPDASVNFSEFSDG

SEQ ID NO: 52 - protein, 44 aa, Artificial Sequence
  Synthetic
MGKGDPKKPRGKMSSYAFFVQTCREEHKKKHPDASVNFSEFSDH

SEQ ID NO: 53 - protein, 44 aa, Artificial Sequence
  Synthetic
MGKGDPKKPRGKMSSYAFFVQTCREEHKKKHPDASVNFSEFSDI

SEQ ID NO: 54 - protein, 44 aa, Artificial Sequence
  Synthetic
MGKGDPKKPRGKMSSYAFFVQTCREEHKKKHPDASVNFSEFSDK

SEQ ID NO: 55 - protein, 44 aa, Artificial Sequence
  Synthetic
MGKGDPKKPRGKMSSYAFFVQTCREEHKKKHPDASVNFSEFSDL

SEQ ID NO: 56 - protein, 44 aa, Artificial Sequence
  Synthetic
MGKGDPKKPRGKMSSYAFFVQTCREEHKKKHPDASVNFSEFSDM

SEQ ID NO: 57 - protein, 44 aa, Artificial Sequence
  Synthetic
MGKGDPKKPRGKMSSYAFFVQTCREEHKKKHPDASVNFSEFSDN

SEQ ID NO: 58 - protein, 44 aa, Artificial Sequence
  Synthetic
MGKGDPKKPRGKMSSYAFFVQTCREEHKKKHPDASVNFSEFSDP

SEQ ID NO: 59 - protein, 44 aa, Artificial Sequence
  Synthetic
MGKGDPKKPRGKMSSYAFFVQTCREEHKKKHPDASVNFSEFSDQ

SEQ ID NO: 60 - protein, 44 aa, Artificial Sequence
  Synthetic
MGKGDPKKPRGKMSSYAFFVQTCREEHKKKHPDASVNFSEFSDR

SEQ ID NO: 61 - protein, 44 aa, Artificial Sequence
  Synthetic
MGKGDPKKPRGKMSSYAFFVQTCREEHKKKHPDASVNFSEFSDS

SEQ ID NO: 62 - protein, 44 aa, Artificial Sequence
  Synthetic
MGKGDPKKPRGKMSSYAFFVQTCREEHKKKHPDASVNFSEFSDT

SEQ ID NO: 63 - protein, 44 aa, Artificial Sequence
  Synthetic
MGKGDPKKPRGKMSSYAFFVQTCREEHKKKHPDASVNFSEFSDV

SEQ ID NO: 64 - protein, 44 aa, Artificial Sequence
  Synthetic
MGKGDPKKPRGKMSSYAFFVQTCREEHKKKHPDASVNFSEFSDW

SEQ ID NO: 65 - protein, 44 aa, Artificial Sequence
  Synthetic
MGKGDPKKPRGKMSSYAFFVQTCREEHKKKHPDASVNFSEFSDY

SEQ ID NO: 66 - protein, 44 aa, Artificial Sequence
  Synthetic
MGKGDPKKPRGKMSSYAFFVQTCREEHKKKHPDASVNFSEFSEA

SEQ ID NO: 67 - protein, 44 aa, Artificial Sequence
  Synthetic
MGKGDPKKPRGKMSSYAFFVQTCREEHKKKHPDASVNFSEFSEC

SEQ ID NO: 68 - protein, 44 aa, Artificial Sequence
  Synthetic
MGKGDPKKPRGKMSSYAFFVQTCREEHKKKHPDASVNFSEFSED

SEQ ID NO: 69 - protein, 44 aa, Artificial Sequence
  Synthetic
MGKGDPKKPRGKMSSYAFFVQTCREEHKKKHPDASVNFSEFSEE

SEQ ID NO: 70 - protein, 44 aa, Artificial Sequence
  Synthetic
MGKGDPKKPRGKMSSYAFFVQTCREEHKKKHPDASVNFSEFSEF

SEQ ID NO: 71 - protein, 44 aa, Artificial Sequence
  Synthetic
MGKGDPKKPRGKMSSYAFFVQTCREEHKKKHPDASVNFSEFSEG

SEQ ID NO: 72 - protein, 44 aa, Artificial Sequence
  Synthetic
MGKGDPKKPRGKMSSYAFFVQTCREEHKKKHPDASVNFSEFSEH

SEQ ID NO: 73 - protein, 44 aa, Artificial Sequence
  Synthetic
MGKGDPKKPRGKMSSYAFFVQTCREEHKKKHPDASVNFSEFSEI

SEQ ID NO: 74 - protein, 44 aa, Artificial Sequence
  Synthetic
MGKGDPKKPRGKMSSYAFFVQTCREEHKKKHPDASVNFSEFSEK

SEQ ID NO: 75 - protein, 44 aa, Artificial Sequence
  Synthetic
MGKGDPKKPRGKMSSYAFFVQTCREEHKKKHPDASVNFSEFSEL

SEQ ID NO: 76 - protein, 44 aa, Artificial Sequence
  Synthetic
MGKGDPKKPRGKMSSYAFFVQTCREEHKKKHPDASVNFSEFSEM

SEQ ID NO: 77 - protein, 44 aa, Artificial Sequence
  Synthetic
MGKGDPKKPRGKMSSYAFFVQTCREEHKKKHPDASVNFSEFSEN

SEQ ID NO: 78 - protein, 44 aa, Artificial Sequence
  Synthetic
MGKGDPKKPRGKMSSYAFFVQTCREEHKKKHPDASVNFSEFSEP

SEQ ID NO: 79 - protein, 44 aa, Artificial Sequence
  Synthetic
MGKGDPKKPRGKMSSYAFFVQTCREEHKKKHPDASVNFSEFSEQ

SEQ ID NO: 80 - protein, 44 aa, Artificial Sequence
  Synthetic
MGKGDPKKPRGKMSSYAFFVQTCREEHKKKHPDASVNFSEFSER

SEQ ID NO: 81 - protein, 44 aa, Artificial Sequence
  Synthetic
MGKGDPKKPRGKMSSYAFFVQTCREEHKKKHPDASVNFSEFSES

SEQ ID NO: 82 - protein, 44 aa, Artificial Sequence
  Synthetic
MGKGDPKKPRGKMSSYAFFVQTCREEHKKKHPDASVNFSEFSET

SEQ ID NO: 83 - protein, 44 aa, Artificial Sequence
  Synthetic
MGKGDPKKPRGKMSSYAFFVQTCREEHKKKHPDASVNFSEFSEV

SEQ ID NO: 84 - protein, 44 aa, Artificial Sequence
  Synthetic
MGKGDPKKPRGKMSSYAFFVQTCREEHKKKHPDASVNFSEFSEW

SEQ ID NO: 85 - protein, 44 aa, Artificial Sequence
  Synthetic
MGKGDPKKPRGKMSSYAFFVQTCREEHKKKHPDASVNFSEFSEY

SEQ ID NO: 86 - protein, 44 aa, Artificial Sequence
  Synthetic
MGKGDPKKPRGKMSSYAFFVQTCREEHKKKHPDASVNFSEFSFA

SEQ ID NO: 87 - protein, 44 aa, Artificial Sequence
  Synthetic
MGKGDPKKPRGKMSSYAFFVQTCREEHKKKHPDASVNFSEFSFC

SEQ ID NO: 88 - protein, 44 aa, Artificial Sequence
  Synthetic
MGKGDPKKPRGKMSSYAFFVQTCREEHKKKHPDASVNFSEFSFD

SEQ ID NO: 89 - protein, 44 aa, Artificial Sequence
  Synthetic
MGKGDPKKPRGKMSSYAFFVQTCREEHKKKHPDASVNFSEFSFE

SEQ ID NO: 90 - protein, 44 aa, Artificial Sequence
  Synthetic
MGKGDPKKPRGKMSSYAFFVQTCREEHKKKHPDASVNFSEFSFF

SEQ ID NO: 91 - protein, 44 aa, Artificial Sequence
  Synthetic
MGKGDPKKPRGKMSSYAFFVQTCREEHKKKHPDASVNFSEFSFG

SEQ ID NO: 92 - protein, 44 aa, Artificial Sequence
  Synthetic
MGKGDPKKPRGKMSSYAFFVQTCREEHKKKHPDASVNFSEFSFH

SEQ ID NO: 93 - protein, 44 aa, Artificial Sequence
  Synthetic
MGKGDPKKPRGKMSSYAFFVQTCREEHKKKHPDASVNFSEFSFI

SEQ ID NO: 94 - protein, 44 aa, Artificial Sequence
  Synthetic
MGKGDPKKPRGKMSSYAFFVQTCREEHKKKHPDASVNFSEFSFK

SEQ ID NO: 95 - protein, 44 aa, Artificial Sequence
  Synthetic
MGKGDPKKPRGKMSSYAFFVQTCREEHKKKHPDASVNFSEFSFL

SEQ ID NO: 96 - protein, 44 aa, Artificial Sequence
  Synthetic
MGKGDPKKPRGKMSSYAFFVQTCREEHKKKHPDASVNFSEFSFM

SEQ ID NO: 97 - protein, 44 aa, Artificial Sequence
  Synthetic
MGKGDPKKPRGKMSSYAFFVQTCREEHKKKHPDASVNFSEFSFN

SEQ ID NO: 98 - protein, 44 aa, Artificial Sequence
  Synthetic
MGKGDPKKPRGKMSSYAFFVQTCREEHKKKHPDASVNFSEFSFP

SEQ ID NO: 99 - protein, 44 aa, Artificial Sequence
  Synthetic
MGKGDPKKPRGKMSSYAFFVQTCREEHKKKHPDASVNFSEFSFQ

SEQ ID NO: 100 - protein, 44 aa, Artificial Sequence
  Synthetic
MGKGDPKKPRGKMSSYAFFVQTCREEHKKKHPDASVNFSEFSFR

SEQ ID NO: 101 - protein, 44 aa, Artificial Sequence
  Synthetic
MGKGDPKKPRGKMSSYAFFVQTCREEHKKKHPDASVNFSEFSFS

SEQ ID NO: 102 - protein, 44 aa, Artificial Sequence
  Synthetic
MGKGDPKKPRGKMSSYAFFVQTCREEHKKKHPDASVNFSEFSFT

SEQ ID NO: 103 - protein, 44 aa, Artificial Sequence
  Synthetic
MGKGDPKKPRGKMSSYAFFVQTCREEHKKKHPDASVNFSEFSFV

SEQ ID NO: 104 - protein, 44 aa, Artificial Sequence
  Synthetic
MGKGDPKKPRGKMSSYAFFVQTCREEHKKKHPDASVNFSEFSFW

SEQ ID NO: 105 - protein, 44 aa, Artificial Sequence
  Synthetic
MGKGDPKKPRGKMSSYAFFVQTCREEHKKKHPDASVNFSEFSFY

SEQ ID NO: 106 - protein, 44 aa, Artificial Sequence
  Synthetic
MGKGDPKKPRGKMSSYAFFVQTCREEHKKKHPDASVNFSEFSGA

SEQ ID NO: 107 - protein, 44 aa, Artificial Sequence
  Synthetic
MGKGDPKKPRGKMSSYAFFVQTCREEHKKKHPDASVNFSEFSGC

SEQ ID NO: 108 - protein, 44 aa, Artificial Sequence
  Synthetic
MGKGDPKKPRGKMSSYAFFVQTCREEHKKKHPDASVNFSEFSGD

SEQ ID NO: 109 - protein, 44 aa, Artificial Sequence
  Synthetic
MGKGDPKKPRGKMSSYAFFVQTCREEHKKKHPDASVNFSEFSGE

SEQ ID NO: 110 - protein, 44 aa, Artificial Sequence
  Synthetic
MGKGDPKKPRGKMSSYAFFVQTCREEHKKKHPDASVNFSEFSGF

SEQ ID NO: 111 - protein, 44 aa, Artificial Sequence
  Synthetic
MGKGDPKKPRGKMSSYAFFVQTCREEHKKKHPDASVNFSEFSGG

SEQ ID NO: 112 - protein, 44 aa, Artificial Sequence
  Synthetic
MGKGDPKKPRGKMSSYAFFVQTCREEHKKKHPDASVNFSEFSGH

SEQ ID NO: 113 - protein, 44 aa, Artificial Sequence
  Synthetic
MGKGDPKKPRGKMSSYAFFVQTCREEHKKKHPDASVNFSEFSGI

SEQ ID NO: 114 - protein, 44 aa, Artificial Sequence
  Synthetic
MGKGDPKKPRGKMSSYAFFVQTCREEHKKKHPDASVNFSEFSGK

SEQ ID NO: 115 - protein, 44 aa, Artificial Sequence
  Synthetic
MGKGDPKKPRGKMSSYAFFVQTCREEHKKKHPDASVNFSEFSGL

SEQ ID NO: 116 - protein, 44 aa, Artificial Sequence
  Synthetic
MGKGDPKKPRGKMSSYAFFVQTCREEHKKKHPDASVNFSEFSGM

SEQ ID NO: 117 - protein, 44 aa, Artificial Sequence
  Synthetic
MGKGDPKKPRGKMSSYAFFVQTCREEHKKKHPDASVNFSEFSGN

SEQ ID NO: 118 - protein, 44 aa, Artificial Sequence
  Synthetic
MGKGDPKKPRGKMSSYAFFVQTCREEHKKKHPDASVNFSEFSGP

SEQ ID NO: 119 - protein, 44 aa, Artificial Sequence
  Synthetic
MGKGDPKKPRGKMSSYAFFVQTCREEHKKKHPDASVNFSEFSGQ

SEQ ID NO: 120 - protein, 44 aa, Artificial Sequence
  Synthetic
MGKGDPKKPRGKMSSYAFFVQTCREEHKKKHPDASVNFSEFSGR

SEQ ID NO: 121 - protein, 44 aa, Artificial Sequence
  Synthetic
MGKGDPKKPRGKMSSYAFFVQTCREEHKKKHPDASVNFSEFSGS

SEQ ID NO: 122 - protein, 44 aa, Artificial Sequence
  Synthetic
MGKGDPKKPRGKMSSYAFFVQTCREEHKKKHPDASVNFSEFSGT

SEQ ID NO: 123 - protein, 44 aa, Artificial Sequence
  Synthetic
MGKGDPKKPRGKMSSYAFFVQTCREEHKKKHPDASVNFSEFSGV

SEQ ID NO: 124 - protein, 44 aa, Artificial Sequence
  Synthetic
MGKGDPKKPRGKMSSYAFFVQTCREEHKKKHPDASVNFSEFSGW

SEQ ID NO: 125 - protein, 44 aa, Artificial Sequence
  Synthetic
MGKGDPKKPRGKMSSYAFFVQTCREEHKKKHPDASVNFSEFSGY

SEQ ID NO: 126 - protein, 44 aa, Artificial Sequence
  Synthetic
MGKGDPKKPRGKMSSYAFFVQTCREEHKKKHPDASVNFSEFSHA

SEQ ID NO: 127 - protein, 44 aa, Artificial Sequence
  Synthetic
MGKGDPKKPRGKMSSYAFFVQTCREEHKKKHPDASVNFSEFSHC

SEQ ID NO: 128 - protein, 44 aa, Artificial Sequence
  Synthetic
MGKGDPKKPRGKMSSYAFFVQTCREEHKKKHPDASVNFSEFSHD

SEQ ID NO: 129 - protein, 44 aa, Artificial Sequence
  Synthetic
MGKGDPKKPRGKMSSYAFFVQTCREEHKKKHPDASVNFSEFSHE

SEQ ID NO: 130 - protein, 44 aa, Artificial Sequence
  Synthetic
MGKGDPKKPRGKMSSYAFFVQTCREEHKKKHPDASVNFSEFSHF

SEQ ID NO: 131 - protein, 44 aa, Artificial Sequence
  Synthetic
MGKGDPKKPRGKMSSYAFFVQTCREEHKKKHPDASVNFSEFSHG

SEQ ID NO: 132 - protein, 44 aa, Artificial Sequence
  Synthetic
MGKGDPKKPRGKMSSYAFFVQTCREEHKKKHPDASVNFSEFSHH

SEQ ID NO: 133 - protein, 44 aa, Artificial Sequence
  Synthetic
MGKGDPKKPRGKMSSYAFFVQTCREEHKKKHPDASVNFSEFSHI

SEQ ID NO: 134 - protein, 44 aa, Artificial Sequence
  Synthetic
MGKGDPKKPRGKMSSYAFFVQTCREEHKKKHPDASVNFSEFSHK

SEQ ID NO: 135 - protein, 44 aa, Artificial Sequence
  Synthetic
MGKGDPKKPRGKMSSYAFFVQTCREEHKKKHPDASVNFSEFSHL

SEQ ID NO: 136 - protein, 44 aa, Artificial Sequence
  Synthetic
MGKGDPKKPRGKMSSYAFFVQTCREEHKKKHPDASVNFSEFSHM

SEQ ID NO: 137 - protein, 44 aa, Artificial Sequence
  Synthetic
MGKGDPKKPRGKMSSYAFFVQTCREEHKKKHPDASVNFSEFSHN

SEQ ID NO: 138 - protein, 44 aa, Artificial Sequence
  Synthetic
MGKGDPKKPRGKMSSYAFFVQTCREEHKKKHPDASVNFSEFSHP

SEQ ID NO: 139 - protein, 44 aa, Artificial Sequence
  Synthetic
MGKGDPKKPRGKMSSYAFFVQTCREEHKKKHPDASVNFSEFSHQ

SEQ ID NO: 140 - protein, 44 aa, Artificial Sequence
  Synthetic
MGKGDPKKPRGKMSSYAFFVQTCREEHKKKHPDASVNFSEFSHR

SEQ ID NO: 141 - protein, 44 aa, Artificial Sequence
  Synthetic
MGKGDPKKPRGKMSSYAFFVQTCREEHKKKHPDASVNFSEFSHS

SEQ ID NO: 142 - protein, 44 aa, Artificial Sequence
  Synthetic
MGKGDPKKPRGKMSSYAFFVQTCREEHKKKHPDASVNFSEFSHT

SEQ ID NO: 143 - protein, 44 aa, Artificial Sequence
  Synthetic
MGKGDPKKPRGKMSSYAFFVQTCREEHKKKHPDASVNFSEFSHV

SEQ ID NO: 144 - protein, 44 aa, Artificial Sequence
  Synthetic
MGKGDPKKPRGKMSSYAFFVQTCREEHKKKHPDASVNFSEFSHW

SEQ ID NO: 145 - protein, 44 aa, Artificial Sequence
  Synthetic
MGKGDPKKPRGKMSSYAFFVQTCREEHKKKHPDASVNFSEFSHY

SEQ ID NO: 146 - protein, 44 aa, Artificial Sequence
  Synthetic
MGKGDPKKPRGKMSSYAFFVQTCREEHKKKHPDASVNFSEFSIA

SEQ ID NO: 147 - protein, 44 aa, Artificial Sequence
  Synthetic
MGKGDPKKPRGKMSSYAFFVQTCREEHKKKHPDASVNFSEFSIC

SEQ ID NO: 148 - protein, 44 aa, Artificial Sequence
  Synthetic
MGKGDPKKPRGKMSSYAFFVQTCREEHKKKHPDASVNFSEFSID

SEQ ID NO: 149 - protein, 44 aa, Artificial Sequence
  Synthetic
MGKGDPKKPRGKMSSYAFFVQTCREEHKKKHPDASVNFSEFSIE

SEQ ID NO: 150 - protein, 44 aa, Artificial Sequence
  Synthetic
MGKGDPKKPRGKMSSYAFFVQTCREEHKKKHPDASVNFSEFSIF

SEQ ID NO: 151 - protein, 44 aa, Artificial Sequence
  Synthetic
MGKGDPKKPRGKMSSYAFFVQTCREEHKKKHPDASVNFSEFSIG

SEQ ID NO: 152 - protein, 44 aa, Artificial Sequence
  Synthetic
MGKGDPKKPRGKMSSYAFFVQTCREEHKKKHPDASVNFSEFSIH

SEQ ID NO: 153 - protein, 44 aa, Artificial Sequence
  Synthetic
MGKGDPKKPRGKMSSYAFFVQTCREEHKKKHPDASVNFSEFSII

SEQ ID NO: 154 - protein, 44 aa, Artificial Sequence
  Synthetic
MGKGDPKKPRGKMSSYAFFVQTCREEHKKKHPDASVNFSEFSIK

SEQ ID NO: 155 - protein, 44 aa, Artificial Sequence
  Synthetic
MGKGDPKKPRGKMSSYAFFVQTCREEHKKKHPDASVNFSEFSIL

SEQ ID NO: 156 - protein, 44 aa, Artificial Sequence
  Synthetic
MGKGDPKKPRGKMSSYAFFVQTCREEHKKKHPDASVNFSEFSIM

SEQ ID NO: 157 - protein, 44 aa, Artificial Sequence
  Synthetic
MGKGDPKKPRGKMSSYAFFVQTCREEHKKKHPDASVNFSEFSIN

SEQ ID NO: 158 - protein, 44 aa, Artificial Sequence
  Synthetic
MGKGDPKKPRGKMSSYAFFVQTCREEHKKKHPDASVNFSEFSIP

SEQ ID NO: 159 - protein, 44 aa, Artificial Sequence
  Synthetic
MGKGDPKKPRGKMSSYAFFVQTCREEHKKKHPDASVNFSEFSIQ

SEQ ID NO: 160 - protein, 44 aa, Artificial Sequence
  Synthetic
MGKGDPKKPRGKMSSYAFFVQTCREEHKKKHPDASVNFSEFSIR

SEQ ID NO: 161 - protein, 44 aa, Artificial Sequence
  Synthetic
MGKGDPKKPRGKMSSYAFFVQTCREEHKKKHPDASVNFSEFSIS

SEQ ID NO: 162 - protein, 44 aa, Artificial Sequence
  Synthetic
MGKGDPKKPRGKMSSYAFFVQTCREEHKKKHPDASVNFSEFSIT

SEQ ID NO: 163 - protein, 44 aa, Artificial Sequence
  Synthetic
MGKGDPKKPRGKMSSYAFFVQTCREEHKKKHPDASVNFSEFSIV

SEQ ID NO: 164 - protein, 44 aa, Artificial Sequence
  Synthetic
MGKGDPKKPRGKMSSYAFFVQTCREEHKKKHPDASVNFSEFSIW

SEQ ID NO: 165 - protein, 44 aa, Artificial Sequence
  Synthetic
MGKGDPKKPRGKMSSYAFFVQTCREEHKKKHPDASVNFSEFSIY

SEQ ID NO: 166 - protein, 44 aa, Artificial Sequence
  Synthetic
MGKGDPKKPRGKMSSYAFFVQTCREEHKKKHPDASVNFSEFSKC

SEQ ID NO: 167 - protein, 44 aa, Artificial Sequence
  Synthetic
MGKGDPKKPRGKMSSYAFFVQTCREEHKKKHPDASVNFSEFSKD

SEQ ID NO: 168 - protein, 44 aa, Artificial Sequence
  Synthetic
MGKGDPKKPRGKMSSYAFFVQTCREEHKKKHPDASVNFSEFSKF

SEQ ID NO: 169 - protein, 44 aa, Artificial Sequence
  Synthetic
MGKGDPKKPRGKMSSYAFFVQTCREEHKKKHPDASVNFSEFSKG

SEQ ID NO: 170 - protein, 44 aa, Artificial Sequence
  Synthetic
MGKGDPKKPRGKMSSYAFFVQTCREEHKKKHPDASVNFSEFSKH

SEQ ID NO: 171 - protein, 44 aa, Artificial Sequence
  Synthetic
MGKGDPKKPRGKMSSYAFFVQTCREEHKKKHPDASVNFSEFSKI

SEQ ID NO: 172 - protein, 44 aa, Artificial Sequence
  Synthetic
MGKGDPKKPRGKMSSYAFFVQTCREEHKKKHPDASVNFSEFSKL

SEQ ID NO: 173 - protein, 44 aa, Artificial Sequence
  Synthetic
MGKGDPKKPRGKMSSYAFFVQTCREEHKKKHPDASVNFSEFSKM

SEQ ID NO: 174 - protein, 44 aa, Artificial Sequence
  Synthetic
MGKGDPKKPRGKMSSYAFFVQTCREEHKKKHPDASVNFSEFSKN

SEQ ID NO: 175 - protein, 44 aa, Artificial Sequence
  Synthetic
MGKGDPKKPRGKMSSYAFFVQTCREEHKKKHPDASVNFSEFSKQ

SEQ ID NO: 176 - protein, 44 aa, Artificial Sequence
  Synthetic
MGKGDPKKPRGKMSSYAFFVQTCREEHKKKHPDASVNFSEFSKR

SEQ ID NO: 177 - protein, 44 aa, Artificial Sequence
  Synthetic
MGKGDPKKPRGKMSSYAFFVQTCREEHKKKHPDASVNFSEFSKS

SEQ ID NO: 178 - protein, 44 aa, Artificial Sequence
  Synthetic
MGKGDPKKPRGKMSSYAFFVQTCREEHKKKHPDASVNFSEFSKT

SEQ ID NO: 179 - protein, 44 aa, Artificial Sequence
  Synthetic
MGKGDPKKPRGKMSSYAFFVQTCREEHKKKHPDASVNFSEFSKV

SEQ ID NO: 180 - protein, 44 aa, Artificial Sequence
  Synthetic
MGKGDPKKPRGKMSSYAFFVQTCREEHKKKHPDASVNFSEFSKW

SEQ ID NO: 181 - protein, 44 aa, Artificial Sequence
  Synthetic
MGKGDPKKPRGKMSSYAFFVQTCREEHKKKHPDASVNFSEFSKY

SEQ ID NO: 182 - protein, 44 aa, Artificial Sequence
  Synthetic
MGKGDPKKPRGKMSSYAFFVQTCREEHKKKHPDASVNFSEFSLA

SEQ ID NO: 183 - protein, 44 aa, Artificial Sequence
  Synthetic
MGKGDPKKPRGKMSSYAFFVQTCREEHKKKHPDASVNFSEFSLC

SEQ ID NO: 184 - protein, 44 aa, Artificial Sequence
  Synthetic
MGKGDPKKPRGKMSSYAFFVQTCREEHKKKHPDASVNFSEFSLD

SEQ ID NO: 185 - protein, 44 aa, Artificial Sequence
  Synthetic
MGKGDPKKPRGKMSSYAFFVQTCREEHKKKHPDASVNFSEFSLE

SEQ ID NO: 186 - protein, 44 aa, Artificial Sequence
  Synthetic
MGKGDPKKPRGKMSSYAFFVQTCREEHKKKHPDASVNFSEFSLF

SEQ ID NO: 187 - protein, 44 aa, Artificial Sequence
  Synthetic
MGKGDPKKPRGKMSSYAFFVQTCREEHKKKHPDASVNFSEFSLG

SEQ ID NO: 188 - protein, 44 aa, Artificial Sequence
  Synthetic
MGKGDPKKPRGKMSSYAFFVQTCREEHKKKHPDASVNFSEFSLH

SEQ ID NO: 189 - protein, 44 aa, Artificial Sequence
  Synthetic
MGKGDPKKPRGKMSSYAFFVQTCREEHKKKHPDASVNFSEFSLI

SEQ ID NO: 190 - protein, 44 aa, Artificial Sequence
  Synthetic
MGKGDPKKPRGKMSSYAFFVQTCREEHKKKHPDASVNFSEFSLK

SEQ ID NO: 191 - protein, 44 aa, Artificial Sequence
  Synthetic
MGKGDPKKPRGKMSSYAFFVQTCREEHKKKHPDASVNFSEFSLL

SEQ ID NO: 192 - protein, 44 aa, Artificial Sequence
  Synthetic
MGKGDPKKPRGKMSSYAFFVQTCREEHKKKHPDASVNFSEFSLM

SEQ ID NO: 193 - protein, 44 aa, Artificial Sequence
  Synthetic
MGKGDPKKPRGKMSSYAFFVQTCREEHKKKHPDASVNFSEFSLN

SEQ ID NO: 194 - protein, 44 aa, Artificial Sequence
  Synthetic
MGKGDPKKPRGKMSSYAFFVQTCREEHKKKHPDASVNFSEFSLP

SEQ ID NO: 195 - protein, 44 aa, Artificial Sequence
  Synthetic
MGKGDPKKPRGKMSSYAFFVQTCREEHKKKHPDASVNFSEFSLQ

SEQ ID NO: 196 - protein, 44 aa, Artificial Sequence
  Synthetic
MGKGDPKKPRGKMSSYAFFVQTCREEHKKKHPDASVNFSEFSLR

SEQ ID NO: 197 - protein, 44 aa, Artificial Sequence
  Synthetic
MGKGDPKKPRGKMSSYAFFVQTCREEHKKKHPDASVNFSEFSLS

SEQ ID NO: 198 - protein, 44 aa, Artificial Sequence
  Synthetic
MGKGDPKKPRGKMSSYAFFVQTCREEHKKKHPDASVNFSEFSLT

SEQ ID NO: 199 - protein, 44 aa, Artificial Sequence
  Synthetic
MGKGDPKKPRGKMSSYAFFVQTCREEHKKKHPDASVNFSEFSLV

SEQ ID NO: 200 - protein, 44 aa, Artificial Sequence
  Synthetic
MGKGDPKKPRGKMSSYAFFVQTCREEHKKKHPDASVNFSEFSLW

SEQ ID NO: 201 - protein, 44 aa, Artificial Sequence
  Synthetic
MGKGDPKKPRGKMSSYAFFVQTCREEHKKKHPDASVNFSEFSLY

SEQ ID NO: 202 - protein, 44 aa, Artificial Sequence
  Synthetic
MGKGDPKKPRGKMSSYAFFVQTCREEHKKKHPDASVNFSEFSMA

SEQ ID NO: 203 - protein, 44 aa, Artificial Sequence
  Synthetic
MGKGDPKKPRGKMSSYAFFVQTCREEHKKKHPDASVNFSEFSMC

SEQ ID NO: 204 - protein, 44 aa, Artificial Sequence
  Synthetic
MGKGDPKKPRGKMSSYAFFVQTCREEHKKKHPDASVNFSEFSMD

SEQ ID NO: 205 - protein, 44 aa, Artificial Sequence
  Synthetic
MGKGDPKKPRGKMSSYAFFVQTCREEHKKKHPDASVNFSEFSME

SEQ ID NO: 206 - protein, 44 aa, Artificial Sequence
  Synthetic
MGKGDPKKPRGKMSSYAFFVQTCREEHKKKHPDASVNFSEFSMF

SEQ ID NO: 207 - protein, 44 aa, Artificial Sequence
  Synthetic
MGKGDPKKPRGKMSSYAFFVQTCREEHKKKHPDASVNFSEFSMG

SEQ ID NO: 208 - protein, 44 aa, Artificial Sequence
  Synthetic
MGKGDPKKPRGKMSSYAFFVQTCREEHKKKHPDASVNFSEFSMH

SEQ ID NO: 209 - protein, 44 aa, Artificial Sequence
  Synthetic
MGKGDPKKPRGKMSSYAFFVQTCREEHKKKHPDASVNFSEFSMI

SEQ ID NO: 210 - protein, 44 aa, Artificial Sequence
  Synthetic
MGKGDPKKPRGKMSSYAFFVQTCREEHKKKHPDASVNFSEFSMK

SEQ ID NO: 211 - protein, 44 aa, Artificial Sequence
  Synthetic
MGKGDPKKPRGKMSSYAFFVQTCREEHKKKHPDASVNFSEFSML

SEQ ID NO: 212 - protein, 44 aa, Artificial Sequence
  Synthetic
MGKGDPKKPRGKMSSYAFFVQTCREEHKKKHPDASVNFSEFSMM

SEQ ID NO: 213 - protein, 44 aa, Artificial Sequence
  Synthetic
MGKGDPKKPRGKMSSYAFFVQTCREEHKKKHPDASVNFSEFSMN

SEQ ID NO: 214 - protein, 44 aa, Artificial Sequence
  Synthetic
MGKGDPKKPRGKMSSYAFFVQTCREEHKKKHPDASVNFSEFSMP

SEQ ID NO: 215 - protein, 44 aa, Artificial Sequence
  Synthetic
MGKGDPKKPRGKMSSYAFFVQTCREEHKKKHPDASVNFSEFSMQ

SEQ ID NO: 216 - protein, 44 aa, Artificial Sequence
  Synthetic
MGKGDPKKPRGKMSSYAFFVQTCREEHKKKHPDASVNFSEFSMR

SEQ ID NO: 217 - protein, 44 aa, Artificial Sequence
  Synthetic
MGKGDPKKPRGKMSSYAFFVQTCREEHKKKHPDASVNFSEFSMS

SEQ ID NO: 218 - protein, 44 aa, Artificial Sequence
  Synthetic
MGKGDPKKPRGKMSSYAFFVQTCREEHKKKHPDASVNFSEFSMT

SEQ ID NO: 219 - protein, 44 aa, Artificial Sequence
  Synthetic
MGKGDPKKPRGKMSSYAFFVQTCREEHKKKHPDASVNFSEFSMV

SEQ ID NO: 220 - protein, 44 aa, Artificial Sequence
  Synthetic
MGKGDPKKPRGKMSSYAFFVQTCREEHKKKHPDASVNFSEFSMW

SEQ ID NO: 221 - protein, 44 aa, Artificial Sequence
  Synthetic
MGKGDPKKPRGKMSSYAFFVQTCREEHKKKHPDASVNFSEFSMY

SEQ ID NO: 222 - protein, 44 aa, Artificial Sequence
  Synthetic
MGKGDPKKPRGKMSSYAFFVQTCREEHKKKHPDASVNFSEFSNA

SEQ ID NO: 223 - protein, 44 aa, Artificial Sequence
  Synthetic
MGKGDPKKPRGKMSSYAFFVQTCREEHKKKHPDASVNFSEFSNC

SEQ ID NO: 224 - protein, 44 aa, Artificial Sequence
  Synthetic
MGKGDPKKPRGKMSSYAFFVQTCREEHKKKHPDASVNFSEFSND

SEQ ID NO: 225 - protein, 44 aa, Artificial Sequence
  Synthetic
MGKGDPKKPRGKMSSYAFFVQTCREEHKKKHPDASVNFSEFSNE

SEQ ID NO: 226 - protein, 44 aa, Artificial Sequence
  Synthetic
MGKGDPKKPRGKMSSYAFFVQTCREEHKKKHPDASVNFSEFSNF

SEQ ID NO: 227 - protein, 44 aa, Artificial Sequence
  Synthetic
MGKGDPKKPRGKMSSYAFFVQTCREEHKKKHPDASVNFSEFSNG

SEQ ID NO: 228 - protein, 44 aa, Artificial Sequence
  Synthetic
MGKGDPKKPRGKMSSYAFFVQTCREEHKKKHPDASVNFSEFSNH

SEQ ID NO: 229 - protein, 44 aa, Artificial Sequence
  Synthetic
MGKGDPKKPRGKMSSYAFFVQTCREEHKKKHPDASVNFSEFSNI

SEQ ID NO: 230 - protein, 44 aa, Artificial Sequence
  Synthetic
MGKGDPKKPRGKMSSYAFFVQTCREEHKKKHPDASVNFSEFSNK

SEQ ID NO: 231 - protein, 44 aa, Artificial Sequence
  Synthetic
MGKGDPKKPRGKMSSYAFFVQTCREEHKKKHPDASVNFSEFSNL

SEQ ID NO: 232 - protein, 44 aa, Artificial Sequence
  Synthetic
MGKGDPKKPRGKMSSYAFFVQTCREEHKKKHPDASVNFSEFSNM

SEQ ID NO: 233 - protein, 44 aa, Artificial Sequence
  Synthetic
MGKGDPKKPRGKMSSYAFFVQTCREEHKKKHPDASVNFSEFSNN

SEQ ID NO: 234 - protein, 44 aa, Artificial Sequence
  Synthetic
MGKGDPKKPRGKMSSYAFFVQTCREEHKKKHPDASVNFSEFSNP

SEQ ID NO: 235 - protein, 44 aa, Artificial Sequence
  Synthetic
MGKGDPKKPRGKMSSYAFFVQTCREEHKKKHPDASVNFSEFSNQ

SEQ ID NO: 236 - protein, 44 aa, Artificial Sequence
  Synthetic
MGKGDPKKPRGKMSSYAFFVQTCREEHKKKHPDASVNFSEFSNR

SEQ ID NO: 237 - protein, 44 aa, Artificial Sequence
  Synthetic
MGKGDPKKPRGKMSSYAFFVQTCREEHKKKHPDASVNFSEFSNS

SEQ ID NO: 238 - protein, 44 aa, Artificial Sequence
  Synthetic
MGKGDPKKPRGKMSSYAFFVQTCREEHKKKHPDASVNFSEFSNT

SEQ ID NO: 239 - protein, 44 aa, Artificial Sequence
  Synthetic
MGKGDPKKPRGKMSSYAFFVQTCREEHKKKHPDASVNFSEFSNV

SEQ ID NO: 240 - protein, 44 aa, Artificial Sequence
  Synthetic
MGKGDPKKPRGKMSSYAFFVQTCREEHKKKHPDASVNFSEFSNW

SEQ ID NO: 241 - protein, 44 aa, Artificial Sequence
  Synthetic
MGKGDPKKPRGKMSSYAFFVQTCREEHKKKHPDASVNFSEFSNY

SEQ ID NO: 242 - protein, 44 aa, Artificial Sequence
  Synthetic
MGKGDPKKPRGKMSSYAFFVQTCREEHKKKHPDASVNFSEFSPA

SEQ ID NO: 243 - protein, 44 aa, Artificial Sequence
  Synthetic
MGKGDPKKPRGKMSSYAFFVQTCREEHKKKHPDASVNFSEFSPC

SEQ ID NO: 244 - protein, 44 aa, Artificial Sequence
  Synthetic
MGKGDPKKPRGKMSSYAFFVQTCREEHKKKHPDASVNFSEFSPD

SEQ ID NO: 245 - protein, 44 aa, Artificial Sequence
  Synthetic
MGKGDPKKPRGKMSSYAFFVQTCREEHKKKHPDASVNFSEFSPE

SEQ ID NO: 246 - protein, 44 aa, Artificial Sequence
  Synthetic
MGKGDPKKPRGKMSSYAFFVQTCREEHKKKHPDASVNFSEFSPF

SEQ ID NO: 247 - protein, 44 aa, Artificial Sequence
  Synthetic
MGKGDPKKPRGKMSSYAFFVQTCREEHKKKHPDASVNFSEFSPG

SEQ ID NO: 248 - protein, 44 aa, Artificial Sequence
  Synthetic
MGKGDPKKPRGKMSSYAFFVQTCREEHKKKHPDASVNFSEFSPH

SEQ ID NO: 249 - protein, 44 aa, Artificial Sequence
  Synthetic
MGKGDPKKPRGKMSSYAFFVQTCREEHKKKHPDASVNFSEFSPI

SEQ ID NO: 250 - protein, 44 aa, Artificial Sequence
  Synthetic
MGKGDPKKPRGKMSSYAFFVQTCREEHKKKHPDASVNFSEFSPL

SEQ ID NO: 251 - protein, 44 aa, Artificial Sequence
  Synthetic
MGKGDPKKPRGKMSSYAFFVQTCREEHKKKHPDASVNFSEFSPM

SEQ ID NO: 252 - protein, 44 aa, Artificial Sequence
  Synthetic
MGKGDPKKPRGKMSSYAFFVQTCREEHKKKHPDASVNFSEFSPN

SEQ ID NO: 253 - protein, 44 aa, Artificial Sequence
  Synthetic
MGKGDPKKPRGKMSSYAFFVQTCREEHKKKHPDASVNFSEFSPQ

SEQ ID NO: 254 - protein, 44 aa, Artificial Sequence
  Synthetic
MGKGDPKKPRGKMSSYAFFVQTCREEHKKKHPDASVNFSEFSPR

SEQ ID NO: 255 - protein, 44 aa, Artificial Sequence
  Synthetic
MGKGDPKKPRGKMSSYAFFVQTCREEHKKKHPDASVNFSEFSPS

SEQ ID NO: 256 - protein, 44 aa, Artificial Sequence
  Synthetic
MGKGDPKKPRGKMSSYAFFVQTCREEHKKKHPDASVNFSEFSPT

SEQ ID NO: 257 - protein, 44 aa, Artificial Sequence
  Synthetic
MGKGDPKKPRGKMSSYAFFVQTCREEHKKKHPDASVNFSEFSPV

SEQ ID NO: 258 - protein, 44 aa, Artificial Sequence
  Synthetic
MGKGDPKKPRGKMSSYAFFVQTCREEHKKKHPDASVNFSEFSPW

SEQ ID NO: 259 - protein, 44 aa, Artificial Sequence
  Synthetic
MGKGDPKKPRGKMSSYAFFVQTCREEHKKKHPDASVNFSEFSPY

SEQ ID NO: 260 - protein, 44 aa, Artificial Sequence
  Synthetic
MGKGDPKKPRGKMSSYAFFVQTCREEHKKKHPDASVNFSEFSQA

SEQ ID NO: 261 - protein, 44 aa, Artificial Sequence
  Synthetic
MGKGDPKKPRGKMSSYAFFVQTCREEHKKKHPDASVNFSEFSQC

SEQ ID NO: 262 - protein, 44 aa, Artificial Sequence
  Synthetic
MGKGDPKKPRGKMSSYAFFVQTCREEHKKKHPDASVNFSEFSQD

SEQ ID NO: 263 - protein, 44 aa, Artificial Sequence
  Synthetic
MGKGDPKKPRGKMSSYAFFVQTCREEHKKKHPDASVNFSEFSQE

SEQ ID NO: 264 - protein, 44 aa, Artificial Sequence
  Synthetic
MGKGDPKKPRGKMSSYAFFVQTCREEHKKKHPDASVNFSEFSQF

SEQ ID NO: 265 - protein, 44 aa, Artificial Sequence
  Synthetic
MGKGDPKKPRGKMSSYAFFVQTCREEHKKKHPDASVNFSEFSQG

SEQ ID NO: 266 - protein, 44 aa, Artificial Sequence
  Synthetic
MGKGDPKKPRGKMSSYAFFVQTCREEHKKKHPDASVNFSEFSQH

SEQ ID NO: 267 - protein, 44 aa, Artificial Sequence
  Synthetic
MGKGDPKKPRGKMSSYAFFVQTCREEHKKKHPDASVNFSEFSQI

SEQ ID NO: 268 - protein, 44 aa, Artificial Sequence
  Synthetic
MGKGDPKKPRGKMSSYAFFVQTCREEHKKKHPDASVNFSEFSQK

SEQ ID NO: 269 - protein, 44 aa, Artificial Sequence
  Synthetic
MGKGDPKKPRGKMSSYAFFVQTCREEHKKKHPDASVNFSEFSQL

SEQ ID NO: 270 - protein, 44 aa, Artificial Sequence
  Synthetic
MGKGDPKKPRGKMSSYAFFVQTCREEHKKKHPDASVNFSEFSQM

SEQ ID NO: 271 - protein, 44 aa, Artificial Sequence
  Synthetic
MGKGDPKKPRGKMSSYAFFVQTCREEHKKKHPDASVNFSEFSQN

SEQ ID NO: 272 - protein, 44 aa, Artificial Sequence
  Synthetic
MGKGDPKKPRGKMSSYAFFVQTCREEHKKKHPDASVNFSEFSQP

SEQ ID NO: 273 - protein, 44 aa, Artificial Sequence
  Synthetic
MGKGDPKKPRGKMSSYAFFVQTCREEHKKKHPDASVNFSEFSQQ

SEQ ID NO: 274 - protein, 44 aa, Artificial Sequence
  Synthetic
MGKGDPKKPRGKMSSYAFFVQTCREEHKKKHPDASVNFSEFSQR

SEQ ID NO: 275 - protein, 44 aa, Artificial Sequence
  Synthetic
MGKGDPKKPRGKMSSYAFFVQTCREEHKKKHPDASVNFSEFSQS

SEQ ID NO: 276 - protein, 44 aa, Artificial Sequence
  Synthetic
MGKGDPKKPRGKMSSYAFFVQTCREEHKKKHPDASVNFSEFSQT

SEQ ID NO: 277 - protein, 44 aa, Artificial Sequence
  Synthetic
MGKGDPKKPRGKMSSYAFFVQTCREEHKKKHPDASVNFSEFSQV

SEQ ID NO: 278 - protein, 44 aa, Artificial Sequence
  Synthetic
MGKGDPKKPRGKMSSYAFFVQTCREEHKKKHPDASVNFSEFSQW

SEQ ID NO: 279 - protein, 44 aa, Artificial Sequence
  Synthetic
MGKGDPKKPRGKMSSYAFFVQTCREEHKKKHPDASVNFSEFSQY

SEQ ID NO: 280 - protein, 44 aa, Artificial Sequence
  Synthetic
MGKGDPKKPRGKMSSYAFFVQTCREEHKKKHPDASVNFSEFSRA

SEQ ID NO: 281 - protein, 44 aa, Artificial Sequence
  Synthetic
MGKGDPKKPRGKMSSYAFFVQTCREEHKKKHPDASVNFSEFSRC

SEQ ID NO: 282 - protein, 44 aa, Artificial Sequence
  Synthetic
MGKGDPKKPRGKMSSYAFFVQTCREEHKKKHPDASVNFSEFSRD

SEQ ID NO: 283 - protein, 44 aa, Artificial Sequence
  Synthetic
MGKGDPKKPRGKMSSYAFFVQTCREEHKKKHPDASVNFSEFSRE

SEQ ID NO: 284 - protein, 44 aa, Artificial Sequence
  Synthetic
MGKGDPKKPRGKMSSYAFFVQTCREEHKKKHPDASVNFSEFSRF

SEQ ID NO: 285 - protein, 44 aa, Artificial Sequence
  Synthetic
MGKGDPKKPRGKMSSYAFFVQTCREEHKKKHPDASVNFSEFSRG

SEQ ID NO: 286 - protein, 44 aa, Artificial Sequence
  Synthetic
MGKGDPKKPRGKMSSYAFFVQTCREEHKKKHPDASVNFSEFSRH

SEQ ID NO: 287 - protein, 44 aa, Artificial Sequence
  Synthetic
MGKGDPKKPRGKMSSYAFFVQTCREEHKKKHPDASVNFSEFSRI

SEQ ID NO: 288 - protein, 44 aa, Artificial Sequence
  Synthetic
MGKGDPKKPRGKMSSYAFFVQTCREEHKKKHPDASVNFSEFSRK

SEQ ID NO: 289 - protein, 44 aa, Artificial Sequence
  Synthetic
MGKGDPKKPRGKMSSYAFFVQTCREEHKKKHPDASVNFSEFSRL

SEQ ID NO: 290 - protein, 44 aa, Artificial Sequence
  Synthetic
MGKGDPKKPRGKMSSYAFFVQTCREEHKKKHPDASVNFSEFSRM

SEQ ID NO: 291 - protein, 44 aa, Artificial Sequence
  Synthetic
MGKGDPKKPRGKMSSYAFFVQTCREEHKKKHPDASVNFSEFSRN

SEQ ID NO: 292 - protein, 44 aa, Artificial Sequence
  Synthetic
MGKGDPKKPRGKMSSYAFFVQTCREEHKKKHPDASVNFSEFSRP

SEQ ID NO: 293 - protein, 44 aa, Artificial Sequence
  Synthetic
MGKGDPKKPRGKMSSYAFFVQTCREEHKKKHPDASVNFSEFSRQ

SEQ ID NO: 294 - protein, 44 aa, Artificial Sequence
  Synthetic
MGKGDPKKPRGKMSSYAFFVQTCREEHKKKHPDASVNFSEFSRR

SEQ ID NO: 295 - protein, 44 aa, Artificial Sequence
  Synthetic
MGKGDPKKPRGKMSSYAFFVQTCREEHKKKHPDASVNFSEFSRS

SEQ ID NO: 296 - protein, 44 aa, Artificial Sequence
  Synthetic
MGKGDPKKPRGKMSSYAFFVQTCREEHKKKHPDASVNFSEFSRT

SEQ ID NO: 297 - protein, 44 aa, Artificial Sequence
  Synthetic
MGKGDPKKPRGKMSSYAFFVQTCREEHKKKHPDASVNFSEFSRV

SEQ ID NO: 298 - protein, 44 aa, Artificial Sequence
  Synthetic
MGKGDPKKPRGKMSSYAFFVQTCREEHKKKHPDASVNFSEFSRW

SEQ ID NO: 299 - protein, 44 aa, Artificial Sequence
  Synthetic
MGKGDPKKPRGKMSSYAFFVQTCREEHKKKHPDASVNFSEFSRY

SEQ ID NO: 300 - protein, 44 aa, Artificial Sequence
  Synthetic
MGKGDPKKPRGKMSSYAFFVQTCREEHKKKHPDASVNFSEFSSA

SEQ ID NO: 301 - protein, 44 aa, Artificial Sequence
  Synthetic
MGKGDPKKPRGKMSSYAFFVQTCREEHKKKHPDASVNFSEFSSC

SEQ ID NO: 302 - protein, 44 aa, Artificial Sequence
  Synthetic
MGKGDPKKPRGKMSSYAFFVQTCREEHKKKHPDASVNFSEFSSD

SEQ ID NO: 303 - protein, 44 aa, Artificial Sequence
  Synthetic
MGKGDPKKPRGKMSSYAFFVQTCREEHKKKHPDASVNFSEFSSE

SEQ ID NO: 304 - protein, 44 aa, Artificial Sequence
  Synthetic
MGKGDPKKPRGKMSSYAFFVQTCREEHKKKHPDASVNFSEFSSF

SEQ ID NO: 305 - protein, 44 aa, Artificial Sequence
  Synthetic
MGKGDPKKPRGKMSSYAFFVQTCREEHKKKHPDASVNFSEFSSG

SEQ ID NO: 306 - protein, 44 aa, Artificial Sequence
  Synthetic
MGKGDPKKPRGKMSSYAFFVQTCREEHKKKHPDASVNFSEFSSH

SEQ ID NO: 307 - protein, 44 aa, Artificial Sequence
  Synthetic
MGKGDPKKPRGKMSSYAFFVQTCREEHKKKHPDASVNFSEFSSI

SEQ ID NO: 308 - protein, 44 aa, Artificial Sequence
  Synthetic
MGKGDPKKPRGKMSSYAFFVQTCREEHKKKHPDASVNFSEFSSK

SEQ ID NO: 309 - protein, 44 aa, Artificial Sequence
  Synthetic
MGKGDPKKPRGKMSSYAFFVQTCREEHKKKHPDASVNFSEFSSL

SEQ ID NO: 310 - protein, 44 aa, Artificial Sequence
  Synthetic
MGKGDPKKPRGKMSSYAFFVQTCREEHKKKHPDASVNFSEFSSM

SEQ ID NO: 311 - protein, 44 aa, Artificial Sequence
  Synthetic
MGKGDPKKPRGKMSSYAFFVQTCREEHKKKHPDASVNFSEFSSN

SEQ ID NO: 312 - protein, 44 aa, Artificial Sequence
  Synthetic
MGKGDPKKPRGKMSSYAFFVQTCREEHKKKHPDASVNFSEFSSP

SEQ ID NO: 313 - protein, 44 aa, Artificial Sequence
  Synthetic
MGKGDPKKPRGKMSSYAFFVQTCREEHKKKHPDASVNFSEFSSQ

SEQ ID NO: 314 - protein, 44 aa, Artificial Sequence
  Synthetic
MGKGDPKKPRGKMSSYAFFVQTCREEHKKKHPDASVNFSEFSSR

SEQ ID NO: 315 - protein, 44 aa, Artificial Sequence
  Synthetic
MGKGDPKKPRGKMSSYAFFVQTCREEHKKKHPDASVNFSEFSSS

SEQ ID NO: 316 - protein, 44 aa, Artificial Sequence
  Synthetic
MGKGDPKKPRGKMSSYAFFVQTCREEHKKKHPDASVNFSEFSST

SEQ ID NO: 317 - protein, 44 aa, Artificial Sequence
  Synthetic
MGKGDPKKPRGKMSSYAFFVQTCREEHKKKHPDASVNFSEFSSV

SEQ ID NO: 318 - protein, 44 aa, Artificial Sequence
  Synthetic
MGKGDPKKPRGKMSSYAFFVQTCREEHKKKHPDASVNFSEFSSW

SEQ ID NO: 319 - protein, 44 aa, Artificial Sequence
  Synthetic
MGKGDPKKPRGKMSSYAFFVQTCREEHKKKHPDASVNFSEFSSY

SEQ ID NO: 320 - protein, 44 aa, Artificial Sequence
  Synthetic
MGKGDPKKPRGKMSSYAFFVQTCREEHKKKHPDASVNFSEFSTA

SEQ ID NO: 321 - protein, 44 aa, Artificial Sequence
  Synthetic
MGKGDPKKPRGKMSSYAFFVQTCREEHKKKHPDASVNFSEFSTC

SEQ ID NO: 322 - protein, 44 aa, Artificial Sequence
  Synthetic
MGKGDPKKPRGKMSSYAFFVQTCREEHKKKHPDASVNFSEFSTD

SEQ ID NO: 323 - protein, 44 aa, Artificial Sequence
  Synthetic
MGKGDPKKPRGKMSSYAFFVQTCREEHKKKHPDASVNFSEFSTE

SEQ ID NO: 324 - protein, 44 aa, Artificial Sequence
  Synthetic
MGKGDPKKPRGKMSSYAFFVQTCREEHKKKHPDASVNFSEFSTF

SEQ ID NO: 325 - protein, 44 aa, Artificial Sequence
  Synthetic
MGKGDPKKPRGKMSSYAFFVQTCREEHKKKHPDASVNFSEFSTG

SEQ ID NO: 326 - protein, 44 aa, Artificial Sequence
  Synthetic
MGKGDPKKPRGKMSSYAFFVQTCREEHKKKHPDASVNFSEFSTH

SEQ ID NO: 327 - protein, 44 aa, Artificial Sequence
  Synthetic
MGKGDPKKPRGKMSSYAFFVQTCREEHKKKHPDASVNFSEFSTI

SEQ ID NO: 328 - protein, 44 aa, Artificial Sequence
  Synthetic
MGKGDPKKPRGKMSSYAFFVQTCREEHKKKHPDASVNFSEFSTK

SEQ ID NO: 329 - protein, 44 aa, Artificial Sequence
  Synthetic
MGKGDPKKPRGKMSSYAFFVQTCREEHKKKHPDASVNFSEFSTL

SEQ ID NO: 330 - protein, 44 aa, Artificial Sequence
  Synthetic
MGKGDPKKPRGKMSSYAFFVQTCREEHKKKHPDASVNFSEFSTM

SEQ ID NO: 331 - protein, 44 aa, Artificial Sequence
  Synthetic
MGKGDPKKPRGKMSSYAFFVQTCREEHKKKHPDASVNFSEFSTN

SEQ ID NO: 332 - protein, 44 aa, Artificial Sequence
  Synthetic
MGKGDPKKPRGKMSSYAFFVQTCREEHKKKHPDASVNFSEFSTP

SEQ ID NO: 333 - protein, 44 aa, Artificial Sequence
  Synthetic
MGKGDPKKPRGKMSSYAFFVQTCREEHKKKHPDASVNFSEFSTQ

SEQ ID NO: 334 - protein, 44 aa, Artificial Sequence
  Synthetic
MGKGDPKKPRGKMSSYAFFVQTCREEHKKKHPDASVNFSEFSTR

SEQ ID NO: 335 - protein, 44 aa, Artificial Sequence
  Synthetic
MGKGDPKKPRGKMSSYAFFVQTCREEHKKKHPDASVNFSEFSTS

SEQ ID NO: 336 - protein, 44 aa, Artificial Sequence
  Synthetic
MGKGDPKKPRGKMSSYAFFVQTCREEHKKKHPDASVNFSEFSTT

SEQ ID NO: 337 - protein, 44 aa, Artificial Sequence
  Synthetic
MGKGDPKKPRGKMSSYAFFVQTCREEHKKKHPDASVNFSEFSTV

SEQ ID NO: 338 - protein, 44 aa, Artificial Sequence
  Synthetic
MGKGDPKKPRGKMSSYAFFVQTCREEHKKKHPDASVNFSEFSTW

SEQ ID NO: 339 - protein, 44 aa, Artificial Sequence
  Synthetic
MGKGDPKKPRGKMSSYAFFVQTCREEHKKKHPDASVNFSEFSTY

SEQ ID NO: 340 - protein, 44 aa, Artificial Sequence
  Synthetic
MGKGDPKKPRGKMSSYAFFVQTCREEHKKKHPDASVNFSEFSVA

SEQ ID NO: 341 - protein, 44 aa, Artificial Sequence
  Synthetic
MGKGDPKKPRGKMSSYAFFVQTCREEHKKKHPDASVNFSEFSVC

SEQ ID NO: 342 - protein, 44 aa, Artificial Sequence
  Synthetic
MGKGDPKKPRGKMSSYAFFVQTCREEHKKKHPDASVNFSEFSVD

SEQ ID NO: 343 - protein, 44 aa, Artificial Sequence
  Synthetic
MGKGDPKKPRGKMSSYAFFVQTCREEHKKKHPDASVNFSEFSVE

SEQ ID NO: 344 - protein, 44 aa, Artificial Sequence
  Synthetic
MGKGDPKKPRGKMSSYAFFVQTCREEHKKKHPDASVNFSEFSVF

SEQ ID NO: 345 - protein, 44 aa, Artificial Sequence
  Synthetic
MGKGDPKKPRGKMSSYAFFVQTCREEHKKKHPDASVNFSEFSVG

SEQ ID NO: 346 - protein, 44 aa, Artificial Sequence
  Synthetic
MGKGDPKKPRGKMSSYAFFVQTCREEHKKKHPDASVNFSEFSVH

SEQ ID NO: 347 - protein, 44 aa, Artificial Sequence
  Synthetic
MGKGDPKKPRGKMSSYAFFVQTCREEHKKKHPDASVNFSEFSVI

SEQ ID NO: 348 - protein, 44 aa, Artificial Sequence
  Synthetic
MGKGDPKKPRGKMSSYAFFVQTCREEHKKKHPDASVNFSEFSVK

SEQ ID NO: 349 - protein, 44 aa, Artificial Sequence
  Synthetic
MGKGDPKKPRGKMSSYAFFVQTCREEHKKKHPDASVNFSEFSVL

SEQ ID NO: 350 - protein, 44 aa, Artificial Sequence
  Synthetic
MGKGDPKKPRGKMSSYAFFVQTCREEHKKKHPDASVNFSEFSVM

SEQ ID NO: 351 - protein, 44 aa, Artificial Sequence
  Synthetic
MGKGDPKKPRGKMSSYAFFVQTCREEHKKKHPDASVNFSEFSVN

SEQ ID NO: 352 - protein, 44 aa, Artificial Sequence
  Synthetic
MGKGDPKKPRGKMSSYAFFVQTCREEHKKKHPDASVNFSEFSVP

SEQ ID NO: 353 - protein, 44 aa, Artificial Sequence
  Synthetic
MGKGDPKKPRGKMSSYAFFVQTCREEHKKKHPDASVNFSEFSVQ

SEQ ID NO: 354 - protein, 44 aa, Artificial Sequence
  Synthetic
MGKGDPKKPRGKMSSYAFFVQTCREEHKKKHPDASVNFSEFSVR

SEQ ID NO: 355 - protein, 44 aa, Artificial Sequence
  Synthetic
MGKGDPKKPRGKMSSYAFFVQTCREEHKKKHPDASVNFSEFSVS

SEQ ID NO: 356 - protein, 44 aa, Artificial Sequence
  Synthetic
MGKGDPKKPRGKMSSYAFFVQTCREEHKKKHPDASVNFSEFSVT

SEQ ID NO: 357 - protein, 44 aa, Artificial Sequence
  Synthetic
MGKGDPKKPRGKMSSYAFFVQTCREEHKKKHPDASVNFSEFSVV

SEQ ID NO: 358 - protein, 44 aa, Artificial Sequence
  Synthetic
MGKGDPKKPRGKMSSYAFFVQTCREEHKKKHPDASVNFSEFSVW

SEQ ID NO: 359 - protein, 44 aa, Artificial Sequence
  Synthetic
MGKGDPKKPRGKMSSYAFFVQTCREEHKKKHPDASVNFSEFSVY

SEQ ID NO: 360 - protein, 44 aa, Artificial Sequence
  Synthetic
MGKGDPKKPRGKMSSYAFFVQTCREEHKKKHPDASVNFSEFSWA

SEQ ID NO: 361 - protein, 44 aa, Artificial Sequence
  Synthetic
MGKGDPKKPRGKMSSYAFFVQTCREEHKKKHPDASVNFSEFSWC

SEQ ID NO: 362 - protein, 44 aa, Artificial Sequence
  Synthetic
MGKGDPKKPRGKMSSYAFFVQTCREEHKKKHPDASVNFSEFSWD

SEQ ID NO: 363 - protein, 44 aa, Artificial Sequence
  Synthetic
MGKGDPKKPRGKMSSYAFFVQTCREEHKKKHPDASVNFSEFSWE

SEQ ID NO: 364 - protein, 44 aa, Artificial Sequence
  Synthetic
MGKGDPKKPRGKMSSYAFFVQTCREEHKKKHPDASVNFSEFSWF

SEQ ID NO: 365 - protein, 44 aa, Artificial Sequence
  Synthetic
MGKGDPKKPRGKMSSYAFFVQTCREEHKKKHPDASVNFSEFSWG

SEQ ID NO: 366 - protein, 44 aa, Artificial Sequence
  Synthetic
MGKGDPKKPRGKMSSYAFFVQTCREEHKKKHPDASVNFSEFSWH

SEQ ID NO: 367 - protein, 44 aa, Artificial Sequence
  Synthetic
MGKGDPKKPRGKMSSYAFFVQTCREEHKKKHPDASVNFSEFSWI

SEQ ID NO: 368 - protein, 44 aa, Artificial Sequence
  Synthetic
MGKGDPKKPRGKMSSYAFFVQTCREEHKKKHPDASVNFSEFSWK

SEQ ID NO: 369 - protein, 44 aa, Artificial Sequence
  Synthetic
MGKGDPKKPRGKMSSYAFFVQTCREEHKKKHPDASVNFSEFSWL

SEQ ID NO: 370 - protein, 44 aa, Artificial Sequence
  Synthetic
MGKGDPKKPRGKMSSYAFFVQTCREEHKKKHPDASVNFSEFSWM

SEQ ID NO: 371 - protein, 44 aa, Artificial Sequence
  Synthetic
MGKGDPKKPRGKMSSYAFFVQTCREEHKKKHPDASVNFSEFSWN

SEQ ID NO: 372 - protein, 44 aa, Artificial Sequence
  Synthetic
MGKGDPKKPRGKMSSYAFFVQTCREEHKKKHPDASVNFSEFSWP

SEQ ID NO: 373 - protein, 44 aa, Artificial Sequence
  Synthetic
MGKGDPKKPRGKMSSYAFFVQTCREEHKKKHPDASVNFSEFSWQ

SEQ ID NO: 374 - protein, 44 aa, Artificial Sequence
  Synthetic
MGKGDPKKPRGKMSSYAFFVQTCREEHKKKHPDASVNFSEFSWR

SEQ ID NO: 375 - protein, 44 aa, Artificial Sequence
  Synthetic
MGKGDPKKPRGKMSSYAFFVQTCREEHKKKHPDASVNFSEFSWS

SEQ ID NO: 376 - protein, 44 aa, Artificial Sequence
  Synthetic
MGKGDPKKPRGKMSSYAFFVQTCREEHKKKHPDASVNFSEFSWT

SEQ ID NO: 377 - protein, 44 aa, Artificial Sequence
  Synthetic
MGKGDPKKPRGKMSSYAFFVQTCREEHKKKHPDASVNFSEFSWV

SEQ ID NO: 378 - protein, 44 aa, Artificial Sequence
  Synthetic
MGKGDPKKPRGKMSSYAFFVQTCREEHKKKHPDASVNFSEFSWW

SEQ ID NO: 379 - protein, 44 aa, Artificial Sequence
  Synthetic
MGKGDPKKPRGKMSSYAFFVQTCREEHKKKHPDASVNFSEFSWY

SEQ ID NO: 380 - protein, 44 aa, Artificial Sequence
  Synthetic
MGKGDPKKPRGKMSSYAFFVQTCREEHKKKHPDASVNFSEFSYA

SEQ ID NO: 381 - protein, 44 aa, Artificial Sequence
  Synthetic
MGKGDPKKPRGKMSSYAFFVQTCREEHKKKHPDASVNFSEFSYC

SEQ ID NO: 382 - protein, 44 aa, Artificial Sequence
  Synthetic
MGKGDPKKPRGKMSSYAFFVQTCREEHKKKHPDASVNFSEFSYD

SEQ ID NO: 383 - protein, 44 aa, Artificial Sequence
  Synthetic
MGKGDPKKPRGKMSSYAFFVQTCREEHKKKHPDASVNFSEFSYE

SEQ ID NO: 384 - protein, 44 aa, Artificial Sequence
  Synthetic
MGKGDPKKPRGKMSSYAFFVQTCREEHKKKHPDASVNFSEFSYF

SEQ ID NO: 385 - protein, 44 aa, Artificial Sequence
  Synthetic
MGKGDPKKPRGKMSSYAFFVQTCREEHKKKHPDASVNFSEFSYG

SEQ ID NO: 386 - protein, 44 aa, Artificial Sequence
  Synthetic
MGKGDPKKPRGKMSSYAFFVQTCREEHKKKHPDASVNFSEFSYH

SEQ ID NO: 387 - protein, 44 aa, Artificial Sequence
  Synthetic
MGKGDPKKPRGKMSSYAFFVQTCREEHKKKHPDASVNFSEFSYI

SEQ ID NO: 388 - protein, 44 aa, Artificial Sequence
  Synthetic
MGKGDPKKPRGKMSSYAFFVQTCREEHKKKHPDASVNFSEFSYK

SEQ ID NO: 389 - protein, 44 aa, Artificial Sequence
  Synthetic
MGKGDPKKPRGKMSSYAFFVQTCREEHKKKHPDASVNFSEFSYL

SEQ ID NO: 390 - protein, 44 aa, Artificial Sequence
  Synthetic
MGKGDPKKPRGKMSSYAFFVQTCREEHKKKHPDASVNFSEFSYM

SEQ ID NO: 391 - protein, 44 aa, Artificial Sequence
  Synthetic
MGKGDPKKPRGKMSSYAFFVQTCREEHKKKHPDASVNFSEFSYN

SEQ ID NO: 392 - protein, 44 aa, Artificial Sequence
  Synthetic
MGKGDPKKPRGKMSSYAFFVQTCREEHKKKHPDASVNFSEFSYP

SEQ ID NO: 393 - protein, 44 aa, Artificial Sequence
  Synthetic
MGKGDPKKPRGKMSSYAFFVQTCREEHKKKHPDASVNFSEFSYQ

SEQ ID NO: 394 - protein, 44 aa, Artificial Sequence
  Synthetic
MGKGDPKKPRGKMSSYAFFVQTCREEHKKKHPDASVNFSEFSYR

SEQ ID NO: 395 - protein, 44 aa, Artificial Sequence
  Synthetic
MGKGDPKKPRGKMSSYAFFVQTCREEHKKKHPDASVNFSEFSYS

SEQ ID NO: 396 - protein, 44 aa, Artificial Sequence
  Synthetic
MGKGDPKKPRGKMSSYAFFVQTCREEHKKKHPDASVNFSEFSYT

SEQ ID NO: 397 - protein, 44 aa, Artificial Sequence
  Synthetic
MGKGDPKKPRGKMSSYAFFVQTCREEHKKKHPDASVNFSEFSYV

SEQ ID NO: 398 - protein, 44 aa, Artificial Sequence
  Synthetic
MGKGDPKKPRGKMSSYAFFVQTCREEHKKKHPDASVNFSEFSYW

SEQ ID NO: 399 - protein, 44 aa, Artificial Sequence
  Synthetic
MGKGDPKKPRGKMSSYAFFVQTCREEHKKKHPDASVNFSEFSYY

SEQ ID NO: 400 - protein, 43 aa, Artificial Sequence
  Synthetic
GKGDPKKPRGKMSSYAFFVQTCREEHKKKHPDASVNFSEFSKK

SEQ ID NO: 401 - protein, 215 aa, Homo sapiens [sequence omitted]

SEQ ID NO: 402 - protein, 44 aa, Artificial Sequence
  Synthetic
MGKGDPKKPRGKMSSYAFFVQTCREEHKKKHPDASVNFSEFSKK

SEQ ID NO: 403 - protein, 43 aa, Artificial Sequence
  Synthetic
GKGDPKKPRGKMSSYAFFVQTCREEHKKKHPDASVNFSEFSPP